(19) United States
(12) Patent Application Publication
(10) Pub. No.: US 2003/0215462 A1
(43) Pub. Date: Nov. 20, 2003

(54) Use of UGT inhibitors to increase bioavailability

(76) Inventors: Vincent J. Wacher, Carlsbad, CA (US); Leslie Z. Benet, Belvedere, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/327,367

(22) Filed: Dec. 20, 2002

Related U.S. Application Data
(60) Provisional application No. 60/342,656, filed on Dec. 21, 2001.

(51) Int. Cl. A61K 35/78; A61K 31/7072; A61K 31/7048; A61K 31/485; A61K 31/4745; A61K 31/7024; A61K 31/16

(52) U.S. Cl. 424/195.18; 424/777; 424/776; 424/778; 424/747; 514/27; 514/23; 514/282; 514/182; 514/49; 514/283; 514/532; 514/546; 514/625

(57) ABSTRACT

Methods for increasing the bioavailability of certain orally administered pharmaceutical compounds by the coadministration of inhibitors of UDP-glucuronosyltransferase (UGT) are disclosed. Particular combinations of UGT inhibitors and pharmaceutical compound are described.

### CROSS-REFERENCE TO RELATED APPLICATION

The present invention is related to and claims priority to U.S. Provisional Patent Application Serial No. 60/342,656, filed Dec. 21, 2001, entitled “Use of UGT inhibitors to increase bioavailability,” which is incorporated herein by reference.

### INTRODUCTION

1. Technical Field

This invention is directed to the field of pharmacology and particularly to the formulation of pharmaceutical compositions for increased bioavailability.

2. Background

Pharmacokinetics is the study of the fate of pharmaceuticals from the time they are ingested until they are eliminated from the body. The sequence of events for an oral composition includes absorption through the various mucosal surfaces, distribution via the blood stream to various tissues, biotransformation in the liver and other tissues, action at the target site, and elimination of drug or metabolites in urine or bile.

Bioavailability of a drug (pharmaceutical composition) following oral dosing is a critical pharmacokinetic determinant which can be approximated by the following formula:

Fₒᵣₐₗ=F_{ABS}×F_{G}×F_{H}

where Fₒᵣₐₗ is the oral bioavailability fraction, which is the fraction of the oral dose that reaches the circulation in an active, unchanged form. Fₒᵣₐₗ is less than 100% of the active ingredient in the oral dose for four reasons: (1) drug is not absorbed out of the gut lumen into the cells of the intestine and is eliminated in the feces; (2) drug is absorbed into the cells of the intestine but back-transported into the gut lumen; (3) drug is biotransformed by the cells of the intestine (to an inactive metabolite); or (4) drug is eliminated by the cells of the liver, either by biotransformation and/or by transport into the bile. Thus, oral bioavailability is the product of the fraction of the oral dose that is absorbed (F_{ABS}), the fraction of the absorbed dose that successfully reaches the blood side of the gastrointestinal tract (F_{G}), and the fraction of the drug in the G1 blood supply that reaches the heart side of the liver (F_{H}). The extent of gut wall absorption, back transport and metabolism, and liver elimination are all subject to wide inter- and intra-individual variability.

Previous investigations arising in the laboratory of one of the present inventors resulted in new understandings of factors involved with bioavailability and in the invention described in U.S. Pat. No. 5,567,592, issued Oct. 22, 1996. The '592 patent describes general methods for increasing bioavailability of oral pharmaceutical compositions and methods for identifying compounds that increase bioavailability. However, although that invention made it possible to investigate a number of classes of compounds not previously thought to be useful in enhancing bioavailability, the actual process of identifying specific classes of compounds that are superior bioenhancers, among those bioenhancers which work to some degree, still remains a process of investigation and discovery. Within many classes of substances identified as showing general bioenhancing effects, there is surprising variance from class member to class member in the extent of each compound's bioenhancing effect, and some compounds that would at first appear to be enhancers of drug bioavailability because of their membership in a generally effective class of compounds, actually are found to be agents that interfere with the bioavailability of drugs, although the mechanism by which such interference takes place is not yet known. In some cases, a single compound or small group of compounds has been found to be particularly potent as a bioenhancer despite resembling in structure other compounds that have less activity or that even reduce bioavailability.

Accordingly, it is important to identify and confirm the identity of individual compounds or classes of compounds that are particularly useful for enhancing bioavailability. For example, U.S. Pat. Nos. 5,665,386; 5,716,928; and 6,121,234 disclose the use of essential oils to enhance bioavailability. U.S. Pat. No. 5,916,566 discloses the use of benzoin gum. U.S. Pat. No. 5,962,522 discloses the use of propyl gallate to increase bioavailability and U.S. Pat. No. 6,180,666 discloses the use of gallic acid esters.

UDP-glucuronosyltransferases (UGTs) are a widely distributed superfamily of enzymes responsible for converting many endogenous substrates and xenobiotics to more polar, water-soluble conjugates for elimination. Many drugs are known to be metabolized by UDP-glucuronosyltransferase (UGT).

The selective estrogen receptor modulator raloxifene (Evista®, Eli Lilly), which is approved for the treatment of osteoporosis and has shown some efficacy in the prevention of breast cancer, is exclusively and extensively metabolized by UGT enzymes, forming glucuronides at both the 4′- and 6-positions of the molecule. The 4′-glucuronide is the predominant metabolite in humans (Knadler et al. 1995. The disposition and metabolism of ¹⁴C-labeled raloxifene in humans [Abstract]. Pharm. Res. 12: S372). The 6-glucuronide appears to be predominant in rats and mice (Dodge et al. 1997. Evaluation of the major metabolites of raloxifene as modulators of tissue selectivity. J. Steroid Biochem. Mol. Biol. 61: 97-106; Lindstrom et al. 1984. Disposition and metabolism of a new benzothiophene antiestrogen in rats, dogs and monkeys. Xenobiotica 14: 841-7). Approximately 60% of an oral raloxifene dose is absorbed from the gastrointestinal tract, however the absolute bioavailability of raloxifene is only 2.0% due to extensive presystemic glucuronidation (Evista® (raloxifene hydrochloride) approved product labeling. 2000. Eli Lilly and Company).

Another highly glucuronidated drug with poor oral bioavailability is the widely used antihypertensive agent labetalol (Trandate®, Glaxo-Wellcome). An oral dose of labetalol is completely absorbed from the gastrointestinal tract, however extensive presystemic glucuronidation results in an absolute oral bioavailability of 25-30% for labetalol (Trandate® (labetalol hydrochloride) approved product labeling. 1998. Glaxo-Wellcome Inc.; Daneshmend et al. 1984. The influence of food on the oral and intravenous pharmacokinetics of a high clearance drug: a study with labetalol. Br. J. Clin. Pharmacol. 14: 73-8; Luke et al. 1992. Bioavailability of labetalol in patients with end stage renal disease. Ther. Drug Monitor. 14: 203-8.) and 11-29% for its major constituent stereoisomer dilevalol (Kramer et al. 1988. Pharmacokinetics and bioavailability of dilevalol in normotensive volunteers. J. Clin. Pharmacol. 28: 644-8; Tenero, et al. 1989. Pharmacokinetics and pharmacodynamics of dilevalol. Clin. Pharmacol. Ther. 46: 648-56.).

The antineoplastic agent irinotecan (CPT-11; Camptosar®, Pharmacia & Upjohn) is a topoisomerase I inhibitor approved for use in the treatment of metastatic cancer of the colon and rectum. Irinotecan is converted by carboxylesterases in vivo to its active metabolite SN-38, which is subsequently glucuronidated by enzymes of the UGT1A family, in particular UGT1A1 (Slatter et al. 2000. Pharmacokinetics, metabolism, and excretion of irinotecan (CPT-11) following iv infusion of [¹⁴C]CPT-11 in cancer patients. Drug Metab. Dispos. 28: 423-33; Sparreboom et al. 1998. Irinotecan (CPT-11) metabolism and disposition in cancer patients. Clin. Cancer Res. 4: 2747-54; Rivory et al. 1997. Pharmacokinetic interrelationships of irinotecan (CPT-11) and its three major plasma metabolites in patients enrolled in phase I/II trials. Clin. Cancer. Res. 3: 1261-6; Iyer et al. 1998. Genetic predisposition to the metabolism of irinotecan (CPT-11). Role of uridinediphosphate glucuronosyl transferase isoform 1A1 in the glucuronidation of its active metabolite (SN-38) by human liver microsomes. J. Clin. Invest. 101: 847-54; Ciotti et al. 1999. Glucuronidation of 7-ethyl-10-hydroxycamptothecin (SN-38) by the human UDP-glucuronosyltransferases encoded at the UGT 1 locus. Biochem. Biophys. Res. Commun. 260: 199-202). A comparison of oral and intravenous irinotecan doses (50 mg/kg) from different studies indicates that the absolute bioavailability of irinotecan (CPT-11 lactone) in cancer patients is approximately 13% (Drengler et al. 1999. Phase I and pharmacokinetic trial of oral irinotecan administered daily for 5 days every 3 weeks in patients with solid tumors. J. Clin. Oncol. 17: 685-96; Rothenberg et al. 1993. Phase I and pharmacokinetic trial of weekly CPT-11. J. Clin. Oncol. 11: 2194-2204).

Preclinical drug interaction studies have shown that administration of valproic acid, an inhibitor of glucuronidation, prior to administration of irinotecan in rats can substantially increase irinotecan levels in vivo (Gupta et al. 1997. Cancer Chemother. Pharmacol. 39: 440). Clinical data describing UGT-mediated drug interactions with irinotecan are not available, however several case reports have documented decreased clearance of SN-38 and lower levels of SN-38 glucuronide in subjects with UGT1A1 deficiencies (Iyer et al. 1999. Phenotype-genotype correlation of in vitro SN-38 (active metabolite of irinotecan) and bilirubin glucuronidation in human liver tissue with UGT1A1 promoter polymorphism. Clin. Pharmacol. Ther. 65: 576-82; Ando et al. 1998. UGT1A1 genotypes and glucuronidation of SN-38, the active metabolite of irinotecan. Ann. Oncol. 9: 845-7).

Zidovudine (Retrovir®, GlaxoSmithKline) is a pyrimidine nucleoside analogue used in the treatment of HIV. Zidovudine is rapidly and almost completely absorbed from the gastrointestinal tract, however it undergoes extensive presystemic glucuronidation such that the absolute oral bioavailability is 65% (range 52-75%) (Retrovir® (zidovudine) tablets. Approved product labeling. 1998. Glaxo-Wellcome Inc; Moore et al. 1995. Pharmacokinetics and bioavailability of zidovudine and its glucuronidated metabolite in patients with human immunodeficiency virus infection and hepatic disease (AIDS Clinical Trials Group protocol 062). Antimicrob. Agents Chemother. 39: 2732-7). Zidovudine is glucuronidated by UGT2B7 to its major metabolite 3′-azido-3′-deoxythymidine-5′-O-β-glucopyranuronosylthymidine (zidovudine-5′-glucuronide, GDZV) (Barbier et al. 2000. 3′-Azido-3′-deoxythymidine (AZT) is glucuronidated by human UDP-glucuronosyltransferase 2B7 (UGT2B7). Drug Metab. Dispos. 28: 497-502). Hepatic glucuronidation of zidovudine varies as much as 10-fold between individuals (Pacifici et al. 1996. Zidovudine glucuronidation in human liver: interindividual variability. Int. J. Clin. Pharmacol. Ther. 34: 329-34), which can result in large interindividual differences in the efficacy of zidovudine therapy.

A number of drug interaction studies have suggested an effect of UGT inhibitors on zidovudine pharmacokinetics. In clinical-studies with HIV-infected patients, co-administration of valproic acid, a UGT2B7 substrate/inhibitor (Trapnell et al. 1998. Glucuronidation of 3′-azido-3′-deoxythymidine (zidovudine) by human liver microsomes: relevance to clinical pharmacokinetic interactions with atovaquone, fluconazole, methadone and valproic acid. Antimicrob. Agents Chemother. 42: 1592-6), caused a 2-fold increase in zidovudine plasma AUC (Lertora et al. 1994. Pharmacokinetic interaction between zidovudine and valproic acid in patients infected with human immunodeficiency virus. Clin. Pharmacol. Ther. 56: 272-8) and a similar increase in cerebrospinal AZT levels (Akula et al. 1997. Am. J. Med. Sci. 313: 244). Probenecid inhibits zidovudine glucuronidation in vitro (Kamali et al. 1992. Influence of probenecid and paracetamol (acetaminophen) on zidovudine glucuronidation in human liver in vitro. Biopharm. Drug Dispos. 13: 403-9) and caused a 2-fold increase in zidovudine levels in AIDS patients and healthy volunteers (Hadaya et al. 1990. Probenecid inhibits the metabolic and renal clearances of zidovudine (AZT) in human volunteers. Pharm. Res. 7: 411-7; Kornhauser et al. 1989. Probenecid and zidovudine metabolism. Lancet 26: 473-5; de Miranda et al. 1989. Alteration of zidovudine pharmacokinetics by probenecid in patients with AIDS or AIDS-related complex. Clin. Pharmacol. Ther. 46: 494-500). This was due primarily to inhibition of glucuronidation, however renal excretion of zidovudine and its glucuronide were also reduced. Modest increases in zidovudine levels have been achieved by coadministration of the UGT inhibitors atovaquone (Lee et al. 1996. Atovaquone inhibits the glucuronidation and increases the plasma concentrations of zidovudine. Clin. Pharmacol. Ther. 59: 14-21) and fluconazole (Brockmeyer et al. 1997. Pharmacokinetic interaction of fluconazole and zidovudine in HIV-positive patients. Eur. J. Med. Res. 2: 377-83; Sahai et al. 1994. Effect of fluconazole on zidovudine pharmacokinetics in patients infected with human immunodeficiency virus. J. Infect. Dis. 169: 1103-7). In clinical studies with AIDS patients treated with zidovudine, treatment with methadone increased the oral zidovudine AUC by at least 29%. It was suggested that methadone was acting to inhibit zidovudine glucuronidation (McCance-Katz, et al. 1998. J. Acq. Immun. Def. Syndr. Hum. Retrovirol. 18: 435).

Coadministration of probenecid doubles steady-state plasma levels of diflunisal, primarily through a 50% reduction in metabolism to both the acyl- and phenol-glucuronides (Macdonald et al. 1995. Effect of probenecid on the formation and elimination kinetics of sulphate and glucuronide conjugates of diflunisal. Eur. J. Clin. Pharmacol. 47: 519-23). A significant pharmacokinetic interaction between indomethacin and diflunisal has also been observed in healthy volunteers, where concomitant diflunisal caused 2- to 5-fold increases in the plasma AUC of indomethacin, with a corresponding 70% decrease in indomethacin clearance (Van Hecken et al. 1989. Pharmacokinetic interaction between indomethacin and diflunisal. Eur. J. Clin. Pharmacol. 36: 507-12). The AUC of diflunisal was unaffected. The large increase in indomethacin levels was attributed almost entirely to inhibition of indomethacin glucuronidation.

Administration of UGT inhibitors may have benefits beyond improved pharmacokinetics and sustained drug levels. Recent studies have identified glucuronidation as a potentially significant pathway by which cancer cells may become resistant to mycophenolic acid (Franklin et al. 1996. Glucuronidation associated with intrinsic resistance to mycophenolic acid in human colorectal carcinoma cells. Cancer Res. 56: 984-7), SN-38 and epirubicin (Brangi et al. 1999. Camptothecin resistance: role of the ATP-binding cassette (ABC), mitoxantrone-resistance half-transporter (MXR), and potential for glucuronidation in MXR-expressing cells. Cancer Res. 59: 5938-5946). Coadministration of a UGT inhibitor can be used to ameliorate cancer cell resistance and improve cancer chemotherapy.

The majority of published UGT-inhibitors are pharmaceutical compounds, however it should be noted that many food components and natural products may also inhibit UGT enzymes. For example, Zhu et al. (J. Steroid. Biochem. Molec. Biol. 1998. 64: 207) reported that a number of flavonoids, including quercitin and naringenin, inhibited the glucuronidation of estradiol and estrone in rat liver microsomes, as did epigallocatechin gallate.

Different compounds may be substrates for one or more different UGT enzymes. As a result, a compound that inhibits the glucuronidation of one substrate does not necessarily prevent the glucuronidation of all UGT substrates. Accordingly, it is important to identify and confirm the activity of individual compounds or classes of compounds that are capable of enhancing bioavailability by inhibiting UGT.

### SUMMARY OF THE INVENTION

This invention is concerned with optimization of drug bioavailability. The invention maximizes drug bioavailability by using UDP-glucuronosyltransferase (UGT) inhibitors, which are also called “bioenhancers” for purposes of this invention.

The invention is carried out by co-administering one or more UGT inhibitors with an oral pharmaceutical compound (drug) or compounds to increase drug bioavailability. The compositions and methods of this invention can be used to increase drug efficacy in humans and other mammals. Although veterinary use is specifically contemplated, the primary use will be in human treatment. Administration schemes include, but are not limited to, use of oral formulations in humans and use of similar formulations for livestock.

Another object of the present invention is to reduce inter-individual variability of the systemic concentrations of the active pharmaceutical compound, as well as intra-individual variability of the systemic concentrations of the pharmaceutical compound being administered.

One embodiment of the invention is a method for increasing the bioavailability of an orally administered pharmaceutical compound, which method comprises orally coadministering to a mammal in need of treatment by the pharmaceutical compound, the pharmaceutical compound and an inhibitor of a. UGTenzyme normally present in the mammal, wherein the pharmaceutical compound is selected from the group consisting of raloxifene, 2-methoxyestradiol, irinotecan, SN-38, estradiol, labetalol, dilevalol, zidovudine (AZT) and morphine, wherein the inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin, the inhibitor being present in an amount sufficient to provide bioavailability of the pharmaceutical compound in the presence of the inhibitor that is greater than the bioavailability of the pharmaceutical compound in the absence of the inhibitor. Preferred combinations of the pharmaceutical compound with particular UGT inhibitor(s) are described herein.

Another embodiment of the invention is a method of formulating an oral pharmaceutical composition, which method comprises admixing a pharmaceutical compound, a pharmaceutical carrier suitable for oral administration, and a UGT inhibitor, wherein the pharmaceutical compound is selected from the group consisting of raloxifene, 2-methoxyestradiol, irinotecan, SN-38, estradiol, labetalol, dilevalol, zidovudine (AZT) and morphine, wherein the inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin, the inhibitor being present in an amount sufficient to provide bioavailability of the pharmaceutical compound in the presence of the inhibitor that is greater than the bioavailability of the pharmaceutical compound in the absence of the inhibitor. Another embodiment of the invention is a pharmaceutical composition produced by this process.

A further embodiment of the invention is a method of increasing bioavailability of the active compound of an existing oral pharmaceutical composition, which method comprises reformulating the existing composition to provide a reformulated oral composition by admixing the pharmaceutical composition with a UGT inhibitor, wherein the active compound in the composition is selected from the group consisting of raloxifene, 2-methoxyestradiol, irinotecan, SN-38, estradiol, labetalol, dilevalol, zidovudine (AZT) and morphine, wherein the inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin, the inhibitor being present in an amount sufficient to provide bioavailability of the active compound when administered in the reformulated composition greater than the bioavailability of the active compound when administered in the existing pharmaceutical composition.

Other aspects of the invention will be apparent from the description herein.

### BRIEF DESCRIPTION OF THE DRAWINGS

FIG. 1 shows a typical glucuronidation reaction. The figure depicts 7-HFC glucuronidation.

FIG. 2 shows an HPLC trace of 7-HFC glucuronidation by human liver microsomes (upper trace). The lower trace shows the HPLC profile in the absence of UDPGA. The unlabeled peak at approximately 6.4 min. in the HPLC trace was not UDPGA-dependent.

FIG. 3 shows a Lineweaver-Burke plot for 7-HFC glucuronidation in human liver microsomes.

FIG. 4 shows an HPLC trace of raloxifene metabolism by human liver microsomes (upper trace) and UGT1A10 (lower trace). Other unidentified peaks in the HPLC trace were from the microsomes and were not UDPGA-dependent.

FIG. 5 shows the substrate-dependence of glucuronidation of raloxifene by human liver microsomes.

FIG. 6 shows the substrate-dependence of formation of raloxifene glucuronide G1 by recombinant UGT enzymes.

FIG. 7 shows the substrate dependence of formation of raloxifene glucuronide G2 by recombinant UGT enzymes.

FIG. 8 shows an HPLC trace from liver microsomal incubations with 2ME2 (upper trace) and E2 (lower trace). Retention times (min): 2-methoxyestradiol-glucuronides MG1=7.9, MG2=8.95; 17-α-ethinylestradiol=11.2, 2ME2=11.5, E2-3-(β-D-glucuronide)=8.0, E2-17-(β-D-glucuronide)=8.5, E2=10.95.

FIG. 9 shows the substrate-dependence of 2ME2 glucuronidation by human liver microsomes. Units are HPLC peak area normalized to internal standard.

FIG. 10 shows plasma levels of raloxifene following administration of oral raloxifene (10 mg/kg) alone or with quercetin or tannic acid (each 50 mg/kg) to female Sprague-Dawley rats.

### DESCRIPTION OF SPECIFIC EMBODIMENTS

UGT Inhibitors Increase Drug Bioavailability

The present invention arises from continued research into the factors affecting drug bioavailability that were described in earlier applications arising from the laboratory of the present inventors. “Drug bioavailability” is defined here as the total amount of drug systemically available over time. The present invention increases drug bioavailability by inhibiting drug biotransformation. The compounds responsible for increased drug bioavailability are UGT inhibitors. The inhibitors described in the present invention are capable of increasing bioavailability by inhibiting UGT enzymes.

In general, the present invention provides a method for increasing the bioavailability of an orally administered pharmaceutical compound by orally administering the pharmaceutical compound to a mammal in need of treatment concurrently with a UGT inhibitor in sufficient amount to provide bioavailability over time of the compound greater than the bioavailability over time of the compound in the absence of the UGT inhibitor. One manner of determining changes in bioavailability is by measuring integrated systemic concentrations over time of the compound in the presence and absence of the UGT inhibitor. Changes in the integrated systemic concentrations over time are indicated by “area under the curve” (AUC) measurements, an accepted pharmacological technique. Particular UGT inhibitors disclosed are epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, clovebud oil, peppermint oil, silibinin, and silymarin. The present inventors have identified new combinations of particular drugs with particular UGT inhibitors that provide for greater bioavailability of the drug than previously described. Many of the UGT inhibitors described herein were not previously known to inhibit UGT. These inhibitors are particularly effective in increasing bioavailability of certain pharmaceutical compounds that are metabolized in vivo primarily or substantially through the UGT pathway. Such compounds include, but are not limited to, raloxifene, 2-methoxyestradiol, irinotecan, SN-38, estradiol, labetalol, dilevalol, zidovudine (AZT) and morphine. A few of the UGT inhibitors described herein were previously known as inhibitors of cytochrome P4503A (CYP3A). For example, U.S. Pat. Nos. 6,004,927; 6,028,054 and 5,229,116, describe quercetin as a CYP3A inhibitor. Benzoin gum, clove oil, peppermint oil, eugenol, geraniol and menthol are disclosed as CYP3A inhibitors in U.S. Pat. Nos. 5,665,386; 5,716,928; 5,916,566 and 6,121,234. Epicatechin gallate, epigallocatechin gallate, gallocatechin gallate, octyl gallate, propyl gallate and tannic acid are disclosed as CYP3A inhibitors in U.S. Pat. Nos. 5,962,522; 6,180,666 or WO 00/51643A1. The CYP3A inhibitors are disclosed as useful for enhancing the oral bioavailability of compounds that are metabolized in vivo via the CYP3A pathway.

Bioavailability Measurements

The increase in drug bioavailability attributable to administration of the UGT inhibitor can be determined by measuring total systemic drug concentrations over time after coadministration of a drug and the UGT inhibitor and after administration of only the drug. The increase in drug bioavailability is defined as an increase in the Area Under the Curve (AUC). AUC is the integrated measure of systemic drug concentrations over time in units of mass-time/volume. The AUC from time zero (the time of dosing) to time infinity (when no drug remains in the body) following the administration of a drug dose is a measure of the exposure of the patient to the drug. When efficacy of the UGT inhibitor is being measured, the amount and form of active drug administered should be the same in both the coadministration of drug and UGT inhibitor and the administration of the drug alone. For instance, administration of 10 mg of drug alone may result in total systemic drug delivered over time (as measured by AUC) of 500 μg.hr/ml. In coadministration (i.e., in the presence of the UGT inhibitor) the systemic drug AUC may increase to 700 μg.hr/ml. If significantly increased drug bioavailability in the presence of the UGT inhibitor is anticipated, drug doses may need to be reduced for safety.

Systemic drug concentrations are measured using standard drug measurement techniques. “Systemic drug concentration” refers to a drug concentration in a mammal's bodily fluids, such as serum, plasma or blood; the term also includes drug concentrations in tissues bathed by the systemic fluids, including the skin. Systemic drug concentration does not include drug concentrations in digestive fluids. The increase in total systemic drug concentrations is one way of defining an increase of drug bioavailability due to coadministration a UGT inhibitor and the drug. For drugs excreted in part unmetabolized in the urine, an increased amount of unchanged drug in the urine will reflect the increase in systemic concentrations.

Characteristics of Drugs Used with UGT Inhibitors

The word “drug” as used herein is defined as a chemical capable of administration to an organism which modifies or alters the organism's physiology. More preferably the word “drug” as used herein is defined as any substance intended for use in the treatment or prevention of disease. Drug includes synthetic and naturally occurring toxins and bioaffecting substances as well as recognized pharmaceuticals, such as those listed in “The Physicians Desk Reference,” 49th edition, 1995, pages 101-338; “Goodman and Gilman's The Pharmacological Basis of Therapeutics” 9th Edition (1996), pages 103-1645 and 1707-1792; and “The United States Pharmacopeia, The National Formulary”, U.S. Pat. No. 23 NF 18 (1995), the compounds of these references being herein incorporated by reference. The term drug also includes compounds that have the indicated properties that are not yet discovered or available in the U.S. The term drug includes pro-active, activated and metabolized forms of drugs. The present invention can be used with drugs consisting of charged, uncharged, hydrophilic, zwitter-ionic, or hydrophobic species, as well as any combination of these physical characteristics. A hydrophobic drug is defined as a drug which in its non-ionized form is more soluble in lipid or fat than in water.

Compounds (or drugs) from a number of classes of compounds can be administered with UGT inhibitors, for example, but not limited to, the following classes: acetanilides, anilides, aminoquinolines, benzhydryl compounds, benzodiazepines, benzofurans, cannabinoids, cyclic peptides, dibenzazepines, digitalis gylcosides, ergot alkaloids, flavonoids, imidazoles, quinolines, macrolides, naphthalenes, opiates (or morphinans), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids. It is preferred that the compounds (or drugs) administered with the UGT inhibitors are metabolized primarily or exclusively by the UGT pathway. Drugs metabolized by UGT include raloxifene, labetalol, irinotecan and its metabolite SN-38, zidovudine, diflunisal, 2-methoxyestradiol, indomethacin, and estradiol, dilevalol, and morphine. Particularly preferred compounds to be administered with UGT inhibitors include raloxifene, 2-methoxyestradiol, irinotecan, SN-38, estradiol, labetalol, dilevalol, zidovudine (AZT), and morphine. More preferred drugs to be administered with a UGT inhibitor include raloxifene, estradiol, 2-methoxyestradiol and zidovudine.

Increased Drug Bioavailability by Inhibition of UGTs

UDP-Zlucuronosyltransferases (UGTs) and Tissue Location

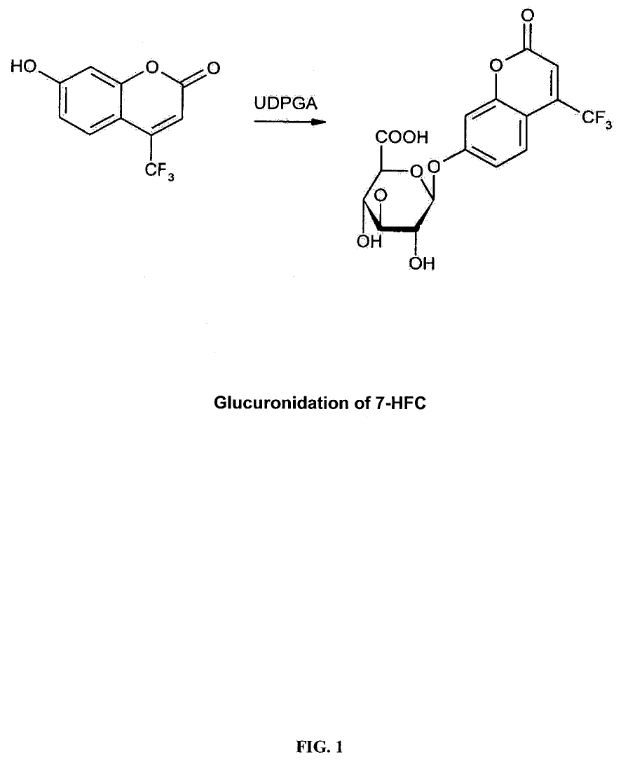

UDP-glucuronosyltransferases (UGTs) are a widely distributed superfamily of enzymes responsible for converting many endogenous substrates and xenobiotics to more polar, water-soluble conjugates for elimination. A typical glucuronidation reaction is illustrated in FIG. 1. In this example, a UGT enzyme catalyzes the reaction of uridine 5′-diphosphoglucuronic acid (UDPGA) with 7-hydroxy-4-(trifluoromethyl)coumarin (7-HFC), resulting in addition of glucuronic acid to the hydroxyl group of the substrate. Glucuronic acid can also be added to carboxylic acid moieties, thiols and amines, and more than one glucuronic acid molecule can be added per substrate molecule. For example, the non-steroidal anti-inflammatory drug diflunisal (Dolobid®, Merck) is glucuronidated at both the acid and hydroxyl substituents (Brunelle, F. M.; Verbeeck, R. K. 1996. Glucuronidation of diflunisal in liver and kidney microsomes of rat and man. Xenobiotica. 26: 123-31).

At least 15 UGT enzyme forms have been identified in humans, with highest concentrations in the liver and tissues of the gastrointestinal tract (See Table 1). Not all of the UGT enzymes are found in all tissues of the body. For example, UGT1A8 mRNA is found in colon but not in the liver, while UGT1A7 appears to be specific to the esophagus and stomach. UGT tissue distribution is further complicated by apparent polymorphic expression in the small intestine. In a recent study, UGT1A1 mRNA was detected in all liver samples studied but was only detected in 1 of 5 jejunum samples and 3 of 5 duodenum and ileum samples (Strassburg, C. P.; Kneip, S.; Topp, J.; Obermayer-Straub, P.; Barut, A.; Tukey, R. H.; Manns, M. P. 2000. Polymorphic gene regulation and interindividual variation of UDP-glucuronosyl transferase activity in human small intestine. J. Biol. Chem. 275: 36164-36171.).- This variability in UGT levels and tissue expression can lead to highly variable absorption and pharmacokinetic profiles for a number of pharmaceuticals.

UGT enzymes in the liver and intestine metabolize a broad range of endogenous molecules, drugs, food constituents and other xenobiotics (See Table 2). UGT-mediated metabolism represents a significant barrier to oral drug absorption, and results in high and variable systemic clearance for a variety of important therapeutic agents. Thus, inclusion of UGT inhibitors in an oral formulation of the UGT substrate drug can result in improved bioavailability, more predictable pharmacokinetics and a better safety profile for these drugs. Also, circulating levels of a UGT inhibitor can help block UGT-mediated resistance in target cells such as tumors, thus improving chemotherapy.

The UGT inhibitors can exert their effects in both the gut and the liver. The location of the UGT inhibitors' effects varies depending on the substrate. As shown in Table 1, there are some gut-specific UGT forms (such as UGT1A10). An inhibitor of UGT1A10 likely produces its effect predominantly in the gut.

The inventors have demonstrated that estradiol and 2-methoxyestradiol have 20-fold greater metabolism in intestinal versus hepatic microsomes. UGT inhibitors that act predominantly in the gut can produce an increase in the oral bioavailability of estradiol and 2-methoxyestradiol.

UGT Inhibitors

By studying the UGT-mediated metabolism of a number of pharmaceutical compounds in human liver and intestinal microsomes as well as microsomes from insect cells transfected with individual human UGT enzyme forms, the present inventors have now identified several combinations of pharmaceutical compounds and UGT inhibitor(s) that can provide improved oral bioavailability of the pharmaceutical compounds.

The ability of a compound to inhibit glucuronidation via the UGT enzymes (and hence the ability to increase to oral bioavailability of a UGT substrate drug) is readily determined by evaluating the inhibition of glucuronidation of a test compound that is a UGT substrate. A convenient test compound for this purpose is 7-hydroxy-4-trifluoromethylcoumarin (7-HFC). 7-HFC is a simple and inexpensive substrate for a number of UGT isozymes including UGT1A1, 1A3, 1A6, 1A7, 1A8, 1A9, 1A10, 2B7, and 2B15. Typically, the inhibition of glucuronidation is determined by incubating the 7-HFC with one or more UGT isozymes, for example from human liver or jejunum microsomes or recombinant UGT enzymes (eg, Supersomes® or Bacculosomes®), in the presence and the absence of the compound to be tested as an inhibitor. Glucuronidation of the 7-HFC substrate is readily determined using HPLC or LC-MS. In addition, or alternatively, UGT substrates other than 7-HFC can be used to assay for inhibitors of glucuronidation. Preferably, one or more of the following UGT substrates are used to assay for inhibitors of glucuronidation: raloxifene, 2-methoxyestradiol, irinotecan, SN-38, estradiol, labetalol, dilevalol, zidovudine (AZT) or morphine.

Using assays similar to those described above, the present inventors have now discovered that tannic acid and octyl gallate are potent inhibitors of 7-HFC glucuronidation in human liver microsomes. In addition, they have shown that lauryl gallate, green tea components epigallocatechin gallate, epicatechin gallate and gallocatechin gallate, ascorbyl palmitate, quercetin, and capsaicin and its analogues inhibit 7-HFC glucuronidation.

Tannic acid and quercetin are demonstrated herein to be good inhibitors of UGT-mediated raloxifene metabolism. Eugenol, silibinin, octyl gallate, and the green tea compounds epicatechin gallate and epigallocatechin gallate are also demonstrated to be good inhibitors of raloxifene metabolism in human liver microsomes. Eugenol and silibinin are the major components of clove oil and silymarin (from Silybum marianum), respectively. Clove oil, silymarin and benzoin powder are shown to be good inhibitors of UGT-mediated raloxifene metabolism.

Glucuronidation of zidovudine is particularly inhibited by tannic acid, gallocatechin gallate, clovebud oil, menthol, menthyl acetate, geraniol, capsaicin and its analogs, and peppermint oil. Estradiol glucuronidation is shown to be particularly inhibited by quercetin, tannic acid, epicatechin gallate, epigallocatechin gallate, gallocatechin gallate and silymarin, as well as allspice berry oil, clovebud oil and peppermint oil. Inhibitors of 2-methoxyestradiol glucuronidation include quercetin, tannic acid, epicatechin gallate, epigallocatechin gallate, gallocatechin gallate, narigenin and peppermint oil.

Particularly preferred UGT inhibitors (that is, bioenhancers) are epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, clovebud oil, peppermint oil, silibinin, and silymarin.

Although most of the compounds that have been tested, as described herein, for their ability to inhibit glucuronidation were previously known to be substrates, or, in a few cases, inhibitors of UGT enzymes, formulation or administration of the preferred compounds together with the particular drugs to increase bioavailability of the drugs was not previously described. In addition, benzoin gum, capsaicin, dihydrocapsaicin, geraniol, and tannic acid have not been identified previously as UGT substrates or inhibitors.

In one embodiment of the present invention, a drug is co-formulated with one or more UGT inhibitors, particularly the preferred UGT inhibitors, to increase the oral bioavailability of the drug.

Increased Drug Efficacy by Inhibiting UGTs

UGT inhibitors reduce drug biotransformation by acting as inhibitors of the activity of the UGT enzymes. Possible mechanisms include competitive, non-competitive, uncompetitive, mixed or irreversible inhibition of UGT-mediated drug biotransformation. Drug biotransformation, as used herein, means metabolism of drugs.

UGT inhibitors, as used according to the invention, reduce drug glucuronidation in the gut by inhibiting UGT activity in the gut which leads to a total increase in drug bioavailability in the serum. In the presence of UGT inhibitors, fewer drug molecules will be metabolized by UGT enzymes in the gut. This will lead to increased concentrations of non-glucuronidated form of the drug passing from the gut into the blood and onto other tissues in the body.

Although the primary objective of the UGT inhibitors is to inhibit UGT-mediated drug biotransformation in the gut, some biotransformation may be decreased in other tissues as well if the UGT inhibitor is absorbed into the blood stream. The decrease in biotransformation by other tissues will also increase drug bioavailability. It is also advantageous if the UGT inhibitors target UGT activity in the liver. After oral administration of UGT inhibitors, concentrations will be highest at the luminal surface of the gut epithelia, not having been diluted by systemic fluids and the tissues of the body. Luminal concentrations that are greater compared to blood concentrations will permit preferential inhibition of UGT activity in the gut.

Coadministration of a UGT inhibitor with a drug will also reduce variability of the oral bioavailability of the drug. Reduction of drug biotransformation or increased drug absorption will decrease variability of oral bioavailability to some degree because the increase in bioavailability will begin to approach the theoretical maximum of 100% oral bioavailability. The increase in oral bioavailability will be generally larger in patients with lower oral bioavailability. The result is a reduction in inter-individual and intra-individual variation. Addition of UGT inhibitors will reduce inter-individual and intra-individual variation of systemic concentrations of a drug or compound.

Selection of UGT Inhibitor Concentration

The ability of the UGT inhibitors to increase oral bioavailability of a particular drug can be estimated using in vitro and in vivo drug biotransformation measurements. In vivo measurements of drug bioavailability, such as measuring serum or blood drug concentrations over time, provide the closest measure of total drug systemic availability. In vitro assays of UGT-mediated metabolism indirectly indicate drug bioavailability because UGT-mediated drug metabolism affects integrated systemic drug concentrations over time. Some in vitro assays of UGT-mediated metabolism are described in the Examples. Although even a minimally measured increase is all that is required for UGT inhibitors to be useful, a preferred commercially desirable concentration of UGT inhibitors generally will increase drug bioavailability by at least 10%, preferably by at least 50%, and more preferably by at least 75% of the difference between bioavailability in its absence and complete oral bioavailability. The term “complete oral bioavailability” as used herein means 100% of the drug is bioavailable when the drug is administered orally. For complete oral bioavailability of a drug, 100% of the drug is present in the patients bodily fluids following oral administration of the drug. Changes in bioavailability are measured against complete oral bioavailability. For example, if the drug bioavailability is 40% without a UGT inhibitor, then the addition of a UGT inhibitor may increase bioavailability to 70%, for a 75% increase. A convenient measure of oral bioavailability is the integrated systemic drug concentrations over time. A sufficient amount of orally administered UGT inhibitor will provide integrated systemic drug concentrations over time greater than the integrated systemic drug concentrations over time in the absence of a UGT inhibitor. The actual amount or concentration of a UGT inhibitor to be included with a pharmaceutical compound for a particular composition or formulation will vary with the active ingredient of the compound. The amount of the UGT inhibitor to be used should be optimized using AUC methods, once the components for a particular pharmaceutical composition have been decided upon. The recommended measure by weight for the amount of a UGT inhibitor in a particular formulation is by direct comparison to the amount of drug, with a UGT inhibitor:drug ratio in the range of 0.01-100:1 being preferred, 0.1-10:1 being more preferred, and 0.5-2:1 being most preferred.

In one embodiment of the invention, the amount of UGT inhibitor used is sufficient to produce a concentration of the inhibitor in the lumen of the gut of the mammal of at least 0.1 times a Kᵢ or apparent Kᵢ of the inhibitor of glucuronidation of the pharmaceutical compound. Kᵢ describes the affinity of a given inhibitor for an enzyme relative to the test substrate (Dixon et al. 1979. “Enzyme inhibition and activation” in Enzymes, 3^{rd} edition. Academic Press. New York. pp: 332-380.).

Inhibition of the UGT enzymes by UGT inhibitors can be studied by a variety of bioassays, several of which are set forth below in the Examples section.

Coadministration and Delivery of UGT Inhibitors

Coadministration of UGT Inhibitor and a Drug

The present invention will increase the bioavailability of a drug in systemic fluids or tissues by co-administering the UGT inhibitor with a drug. “Co-administration” includes concurrent administration (administration of the UGT inhibitor and drug at the same time) and time-varied administration (administration of the UGT inhibitor at a time different from that of the drug), as long as both the UGT inhibitor and the drug are present in the gut lumen and/or membranes during at least partially overlapping times. “Systemic fluids or tissues” refers to blood, plasma, or serum and to other body fluids or tissues in which drug measurements can be obtained.

Delivery Vehicles and Methods

Coadministration can occur with the same delivery vehicle or with different delivery vehicles. The UGT inhibitor and the drug can be administered using, as examples, but not limited to, time release matrices, time release coatings, companion ions, and successive oral administrations. Alternatively, the drug and the UGT inhibitor can be separately formulated with different coatings possessing different time constants for release of UGT inhibitor and drug. UGT inhibitor can also be bound to the drug being protected, either by covalent bonding or by ionic or polar attractions.

Formulations Having a UGT Inhibitor

The invention is carried out in part by formulating an oral or intravenous pharmaceutical composition to contain a UGT inhibitor. This is accomplished in some embodiments by admixing a pharmaceutical compound, usually with a pharmaceutical carrier, and a UGT inhibitor, to form a composition, the UGT inhibitor being present in an amount sufficient to provide bioavailability of the compound (as measured by AUCs or otherwise as described herein) greater than the bioavailability of the compound in the absence of the UGT inhibitor when the pharmaceutical composition is administered orally to an animal being treated. A pharmaceutical carrier is generally an inert bulk agent added to make the active ingredients easier to handle and can be solid or liquid in the usual manner as is well understood in the art. Pharmaceutical compositions produced by the process described herein are also part of the present invention.

The present invention can also be used to increase the bioavailability of the active compound of an existing oral pharmaceutical composition. When practiced in this manner, the invention is carried out by reformulating the existing composition to provide a reformulated composition by admixing the active compound with a UGT inhibitor, the UGT inhibitor being present in an amount sufficient to provide integrated systemic concentrations over time of the active compound when administered in the reformulated composition greater than the integrated systemic concentrations over time of the compound when administered in the existing pharmaceutical composition. All of the criteria described for new formulations also apply to reformulation of old compositions. In preferred aspects of reformulations, the reformulated composition comprises all components present in the existing pharmaceutical composition plus the UGT inhibitor, thus simplifying practice of the invention, although it is also possible to eliminate existing components of formulations because of the increase in bioavailability. Thus, the invention also covers reformulated compositions that contain less than all components present in the existing pharmaceutical composition plus the UGT inhibitor. However, this invention does not cover already-existing compositions that contain a component that increases bioavailability by mechanisms described in this specification (without knowledge of the mechanisms), should such compositions exist.

Traditional formulations can be used with a UGT inhibitor. Optimal UGT inhibitor concentrations can be determined by varying the amount and timing of UGT inhibitor administration and monitoring bioavailability. Once the optimal UGT inhibitor concentration or UGT inhibitor to drug ratio is established for a particular drug, the formulation (UGT inhibitor, drug, and other formulation components, if any) is tested clinically to verify the increased bioavailability. In the case of time- or sustained- release formulations, it will be preferred to establish the optimal UGT inhibitor concentration using such formulations from the start of the bioavailability experiments.

The following examples are illustrative only and are not intended as a limitation on the invention.

### EXAMPLES

Many compounds were evaluated for their activity as inhibitors of UGT enzymes. Table 2 is a compilation of information available in the scientific literature regarding the availability of a number of compounds as substrates for one or more of the UGT isozymes. Such compounds may be effective inhibitors of UGT-mediated metabolism for use as bioavailability enhancers.

The test substrates used in the evaluations described herein were raloxifene, estradiol, 2-methoxyestradiol, zidovudine (AZT) and 7-HFC. 7-HFC is a simple and inexpensive substrate for a broad range of UGT forms including UGT1 A1, 1A3, 1A6, 1A7, 1A8, 1 A9, 1A10, 2B7, and 2B15.

Materials and Methods For Examples

Materials

7-Hydroxy-4-trifluoromethylcoumarin (7-HFC, lot 202) was purchased from Gentest Corp. (Woburn, Mass.). Raloxifene was purchased from Toronto Research Chemicals Inc. (Toronto, Canada). Zidovudine (lot # 120K1334), zidovudine-5′-glucuronide (lot # 59H3872), 17-β-estradiol (lot 79H0940), 17-α-ethinylestradiol (lot 45H0716), β-estradiol-3-(β-D-glucuronide), Na salt (lot 12H3797), β-estradiol-17-(β-D-glucuronide), Na salt (lot 80K3818), uridine 5′-diphosphoglucuronic acid (UDPGA; lot 60H7225), β-glucuronidase (EC 3.2.1.31, Type L-H from limpets; lot 20K3796), and labetalol hydrochloride (lot 105H0123) were purchased from Sigma Chemical Co. (St. Louis Mo.). General laboratory chemicals, substrates and reagents were purchased from Sigma, Aldrich, ICN Biomedicals (Costa Mesa, Calif.), Calbiochem-Novabiochem (La Jolla, Calif.) and Spectrum (Gardena, Calif.).

Analytical Instrumentation

HPLC-UV analysis utilized a Beckman model 126 binary solvent module with detection using a Beckman model 166 Wv detector. Samples were injected using a Beckman model 507e autosampler fitted with a Rheodyne model 7010 sample injection valve (100 μl sample loop volume). Data were collected and analyzed using Beckman System Gold Nouveau™ chromatography software.

HPLC-MS analysis utilized a Hewlett Packard Series 1100 chromatography system with detection using a Series 1100 MSD. Samples were injected using a Series 1100 autosampler fitted with a Rheodyne model 7750-044 sample injection valve (100 μl sample loop volume). Data were collected and analyzed using Hewlett-Packard LC/MSD ChemStation chromatography software.

Human Liver Microsomes

Human liver pieces and pooled human jejunum microsomes were obtained from Tissue Transformation Technologies (Edison, N.J.). Liver pieces were homogenized in 0.1 mM Tris-acetate pH 7.4 containing 1 mM EDTA and 20 mM BHT. Microsomal pellets (lots 021700, 062900, 083000) were prepared using standard differential centrifugation procedures, (Guengerich F. P. Analysis and Characterization of Enzymes in Principles and Methods of Toxicology. A. W. Hayes (ed.), Raven Press. New York. pp: 774-814; 1989) and were stored at −80° C. in Tris-acetate buffer pH 7.4 containing 20% w/v glycerol. Microsomal protein and CYP content were determined using the methods of Bradford (A rapid and sensitive method for the quantitation of microgram quantities of protein using the principles of protein-dye binding. Anal. Biochem. 1976 72: 248-54), and Omura and Sato (The carbon monoxide-binding pigment of liver microsomes II. Solublization, purification and properties. J. Biol. Chem. 1964 239: 2370-8) respectively. Donor profiles are reported in Table A1.

Recombinant UGT Enzymes

Supersomes® containing human UGT1A1, 1A3, 1A4, 1A6, 1A8, 1A9, 1A10, 2B7 and 2B15 were obtained from Gentest Corp. (Woburn, Mass.). Bacculosomes® containing human UGT1A1, 1A3, 1A6, 1A7, 1A8, 1A9, 1A10 and 2B7 were obtained from Panvera Corporation (Madison, Wis.).

### Example 1

### Metabolism Studies with 7-HFC

7-HFC Incubations in Human Liver Microsomes

7-HFC substrate (50 μM; 2 μl of an acetonitrile stock solution) and inhibitor (2 μl of a methanol stock solution) or vehicle were pre-incubated with human liver microsomes (lots 021700, 062900, 062101; 100 μg/ml ) or pooled human jejunum microsomes (100 μg/ml) and 10 mM MgCl₂ in 100 mM Tris HCl buffer (pH 7.5) for 5 min at 37° C. Metabolic reactions were started by addition of UDPGA to give a final UDPGA concentration of 1 mM and a total reaction volume of 200 μl. Reactions were stopped after 15 min at 37° C. by addition of 100 μl stop solution (94:6 acetonitrile-glacial acetic acid) containing naproxen internal standard (500 μM). Samples were vigorously vortex mixed then protein was precipitated by centrifugation (3000 rpm×10 min). All experiments were conducted in triplicate and compared to reactions with inhibitor and substrate but without UDPGA. Possible interfering peaks in the HPLC traces were identified by analysis of metabolic incubations with and without UDPGA in the absence of substrate. Inhibitor efficacy was confirmed in repeated experiments.

Metabolism Kinetics and Kᵢ Studies

Metabolism kinetic studies and Kᵢ determinations measured 7-HFC metabolism over a 25 fold concentration range (20, 50, 100, 200, 500 μM) using the standardized incubation conditions described above for 7-HFC incubations in human liver microsomes. Inhibitor concentrations were 50, 100 and 200 μM for octyl gallate, epigallocatechin gallate and gallocatechin gallate; 2, 5, 10 and 25 μM for tannic acid; 5, 10, 25 and 50 μM for diethylstilbestrol; and 100, 200 and 500 μM for diflunisal and diclofenac. Experiments were conducted in duplicate and data were fit to Michaelis-Menten kinetics by non-linear regression using SigmaPlot® v4.0 (SPSS Inc., San Rafael, Calif.) (Dixon et al. 1979. “Enzyme inhibition and activation” in Enzymes, 3^{rd} edition. Academic Press. New York. pp: 332-380.).

7-HFC Incubations with Recombinant UGT Enzymes

7-HFC substrate (50 μM; 2 μl of a methanol stock solution) was pre-incubated with Supersomes® or Bacculosomes® containing recombinant human UGT enzymes (250 μg/ml microsomal protein) and 10 mM MgCl₂ in 100 mM Tris HCl buffer (pH 7.5) for 5 min at 37° C. Metabolic reactions were started by addition of UDPGA to give a final UDPGA concentration of 1 mM and a total reaction volume of 200 μl. Reactions were stopped after 15 min at 37° C. by addition of 100 μl stop solution (94:6 acetonitrile-glacial acetic acid) containing naproxen internal standard (500 μM) then extracted and analyzed as described above.

Analysis of 7-HFC Incubation Mixtures

7-HFC and its glucuronidation product were separated on a Rainin Microsorb-MV™ C-18 analytical column (5 μm; 4.6×250 mm). Compounds were eluted using a binary solvent gradient system. Solvent A was 70:30 dilute phosphoric acid (pH 3)-acetonitrile. Solvent B was methanol. Solvent flow rate was 1.0 ml/min and the column temperature was maintained at 50° C. Detection was at 325 nm. The initial mobile phase was 80% A and 20% B. Immediately upon sample injection the concentration of B was increased linearly over 20 min to a final concentration of 80% B at which time the system was returned to the initial conditions. Retention times were 3.7 min for 7-HFC glucuronide, 9.9 min for 7-HFC and 10.5 min for naproxen internal standard.

LC-MS analysis of microsomal incubation mixtures utilized a Rainin Microsorb-MV™ C-18 analytical column (5 μm; 4.6 mm×150 mm). Compounds were eluted using the same solvent gradient as above except that the aqueous phase was 1 mM sodium formate (pH 3). Retention times were 7.9 min for 7-HFC glucuronide and 11.9 min for 7-HFC. Sodium adducts of 7-HFC and its glucuronide were analyzed by ESI mass spectrometry using scan ion monitoring. The mass spectrometer was run in the positive ion mode with N₂ drying gas flow of 12 L/min, drying gas temperature 350° C., nebulizer pressure 50 psig, chamber current 0.59 μA, capillary current 31 nA and capillary voltage (V_{cap}) 4000 V.

Quantitation of 7-HFC Glucuronide

A de facto standard curve for 7-HFC glucuronide was generated by metabolizing 7-HFC (2, 5, 10, 20, 50, 100 μM) with human liver microsomes (100 μg/ml) or Gentest UGT1A6 Supersomes® (250 μg/ml) until the substrate had completely disappeared (60 and 120 min). Experiments were conducted in triplicate and compared to duplicate samples where UDPGA was omitted. Standard curves generated for 7-HFC and the glucuronide were linear over the concentration range tested (r²≧0.99) and were superimposable. Moreover, standard curves generated from metabolism in liver microsomes and UGT1A6 Supersomes® were identical. The HPLC peak areas (normalized to the internal standard) of the glucuronide and the unmetabolized 7-HFC were identical for each concentration, indicating that standard curves of 7-HFC can be used to quantitate glucuronide levels.

β-Glucuronidase Cleavage of 7-HFC Glucuronide

7-HFC substrate (50 μM; 10 μl of an acetonitrile stock solution) was pre-incubated with 100 μg/ml human liver microsomal protein (lot 062900) and 10 mM MgCl₂ in 100 M Tris HCl buffer (pH 7.5) for 5 min at 37° C. Metabolic reactions were started by addition of UDPGA to give a final UDPGA concentration of 1 mM and a total reaction volume of 2 ml. After 30 min at 37° C., 200 μl of the incubation mixture was removed and extracted and analyzed as above. The remaining incubation mixture was divided into two equal 0.8 ml samples, one of which was added to 0.8 ml of 100 mM sodium acetate buffer (pH 4.5) while the other 0.8 ml was added to 0.8 ml 100 mM sodium acetate buffer (pH 4.5) containing 1000 units/ml of β-glucuronidase enzyme (Sekikawa, et al. 1995). Apparent intramolecular acyl migration and hydrolysis of furosemide glucuronide in aqueous solution (Biol. Pharm. Bull. 18: 134-9). The mixtures were incubated at 37° C. and 200 μl samples were extracted and analyzed at 0.5, 1, 2, 3 and 4 hr. All experiments were conducted in triplicate.

Results—7-HFC Glucuronidation by Human Liver Microsomes

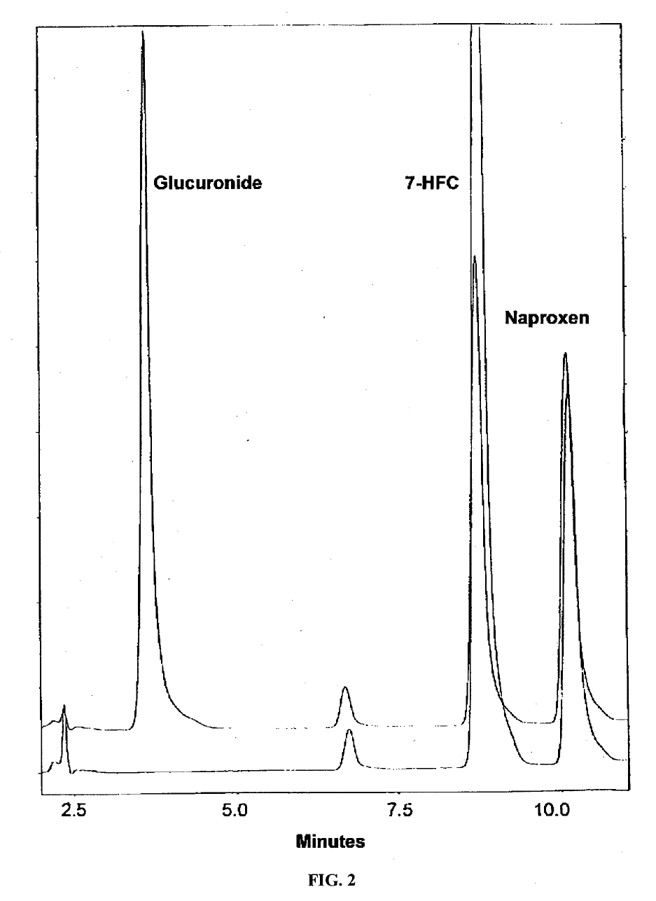

Microsomal metabolism of 7-HFC resulted in one UDPGA-dependent metabolite peak with a retention time of 3.7 min (FIG. 2). A second unidentified peak in the incubation mixture (RT 6.4 min) appears to come from the extraction solvent and did not vary with substrate, UDPGA concentration, microsomal protein concentration or incubation time. LC-MS analysis demonstrated that the molecular weight of the 7-HFC metabolite peak (M—Na⁺ m/z=429.0), was 176 units higher than 7-HFC (M—Na⁺ m/z=253.1), consistent with addition of glucuronic acid. Metabolism was not sensitive to organic solvents and mean metabolism rates in the presence of acetontrile, DMSO and methanol were 90%, 94% and 97% respectively of incubations with buffer alone.

Further confirmation that the metabolite at 3.7 min was a glucuronide adduct was achieved by incubation of glucuronide-containing microsomal incubation mixtures with β-glucuronidase enzyme. In the presence of β-glucuronidase, 7-HFC glucuronide was quantitatively reverted to 7-HFC parent within 30 min. Linear loss of glucuronide and corresponding formation of 7-HFC was also observed in the acetate buffer without β-glucuronidase. The calculated half-lives for hydrolytic loss of glucuronide and consequent formation of 7-HFC in acetate buffer at 37° C. were 6.8 hr and 7.0 hr respectively.

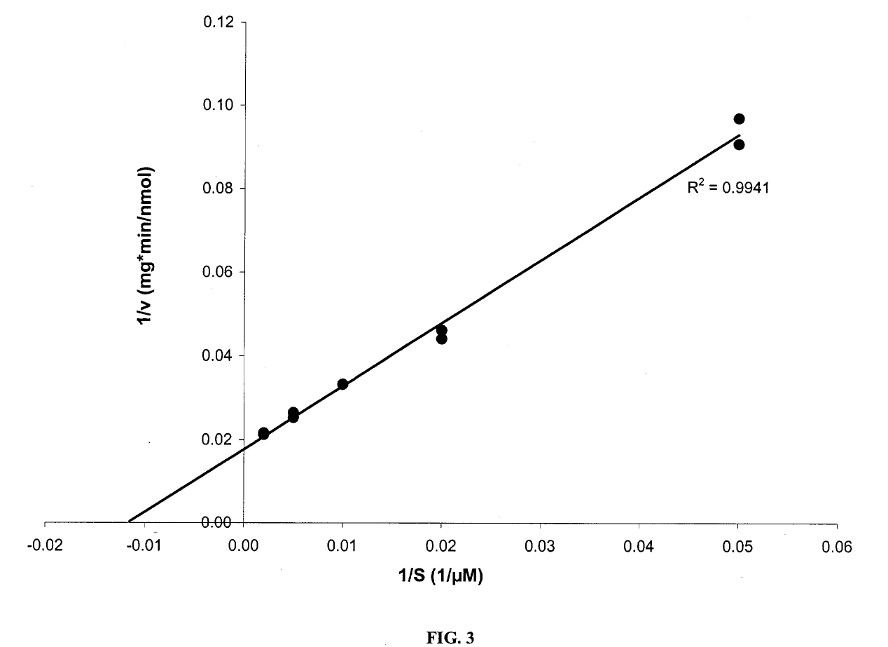

Liver microsomal incubation conditions were optimized by measuring the time course of 7-HFC-glucuronide formation at different protein and UDPGA concentrations. Standardized incubations used 100 μg/ml microsomal protein, 1 mM UDPGA and a 15 min incubation time. Under these conditions, the 7-HFC glucuronidation rates (mean±sd) for three liver microsome lots were 26,405±272, 29,907±562 and 33,039±493 pmol/min/mg (microsomes lots 062900, 032101 and 062101, respectively). Although 7-HFC is a substrate for multiple UGT enzyme forms (Table 3), 7-HFC glucuronidation by microsome lot 062900 was well fit by one-enzyme Michaelis-Menten kinetics over the substrate concentration range tested (20-500 μM) (FIG. 3) and Eadie-Hofstee plots were linear consistent with one dominant metabolizing enzyme in the microsomes used. The mean apparent Kₘ and Vₘₐₓ for 7-HFC glucuronidation in this system were 85 μM and 60 nmol/min/mg microsomal protein.

7-HFC metabolism was also measured in pooled human jejunum microsomes. Using a 50 μM substrate concentration and 100 μg/ml microsomal protein, 7-HFC metabolism was linear for at least 20 min. The formation rate of the glucuronide (mean±sd) was 6,829±61 pmol/min/mg protein, which is 4-fold lower than observed in human liver microsomes.

Results—7-HFC Glucuronidation by Recombinant UGT Enzymes

7-HFC glucuronidation was measured in insect cell microsomes expressing recombinant UGT1A1, 1A3, 1A4, 1A6, 1A7, 1A8, 1A9, 1A10, 2B7 and 2B15. 7-HFC was a substrate for all of these enzymes except UGT1A4 (Table 3). UGT1A6 was the primary 7-HFC metabolizing enzyme for both Gentest Supersomes® and Panvera Bacculosomes®. Significant 7-HFC glucuronidation was also observed with UGT1A9 (81% of the rate for UGT1A6). The Gentest Supersomes® demonstrated significantly higher metabolic activity than Panvera Bacculosomes®. 7-HFC glucuronidation rates for Panvera Bacculosomes® containing UGT1A1, 1A3, 1A6, and 2B7 were 42%, 2%, 31%, and 22% of the respective rates in Gentest Supersomes® using identical conditions. Metabolism rates were approximately equal to UGT1A10 from both sources. No metabolism was observed in untransfected insect microsomes from either supplier. The 7-HFC glucuronidation rate in human liver microsomes was 6-times higher than observed for UGT1A6 Supersomes® (Table 3).

Results—Inhibition of 7-HFC Glucuronidation in Human Liver Microsomes

Standard inhibition screens utilized 50 μM substrate, 1 mM UDPGA and a 15 min incubation time. Data comparing the inhibition of 7-HFC metabolism in human liver microsomes by the UGT inhibitors of the invention, endogenous UGT substrates and drug substrates or inhibitors are presented in Table 4. Complete data for all drugs, endogenous substrates and GRAS compounds and food additives tested are included in Table A2. Tannic acid and octyl gallate were the best inhibitors of 7-HFC glucuronidation in human liver microsomes with IC₅₀ values of 10 μM and approximately 75 μM respectively. The IC₅₀ of tannic acid was an order of magnitude lower than all other inhibitors tested. Other compounds that significantly inhibited 7-HFC glucuronidation included lauryl gallate (IC₅₀=100 μM), the green tea components epigallocatechin gallate (100-200 μM) and gallocatechin gallate (100 μM), ascorbyl palmitate (200 μM), and capsaicin and its analogues (100-200 μM).

Results—Kᵢ Determinations

7-HFC glucuronidation by human liver microsomes was fit by one-enzyme Michaelis-Menten kinetics over the substrate range tested, allowing the determination of an apparent Kᵢ for the best inhibitors (Table 5). Of the compounds tested, diflunisal was the only competitive inhibitor of 7-HFC glucuronidation. Tannic acid was a potent non-competitive glucuronidation inhibitor. Diethylstilbestrol, diclofenac and epigallocatechin gallate were mixed-type inhibitors. Octyl gallate and gallocatechin gallate were mixed-type inhibitors at lower concentrations and strictly non-competitive inhibitors at higher concentrations.

### Example 2

### Metabolism Studies with Raloxifene

Raloxifene Incubations with Human Liver Microsomes

Raloxifene substrate (50 μM; 5 μl of a methanol stock solution) and inhibitor (5 μl of a methanol stock solution) or vehicle were pre-incubated with 250 μg/ml human liver microsomal protein (lots 083000, 032101, 062101) or 100 μg/ml pooled jejunum microsomes and 10 mM MgCl₂ in 100 mM Tris HCl buffer (pH 7.5) for 5 min at 37° C. Metabolic reactions were started by addition of UDPGA to give a final UDPGA concentration of 1 mM and a total reaction volume of 0.5 ml. Reactions were stopped after 15 min at 37° C. by addition of 200 μl stop solution (80% acetonitrile 20% Tris base) containing diethylstilbestrol (100 μM) or nifedipine (100 μM) internal standard. Samples were vigorously vortex mixed then protein was precipitated by centrifugation (3000 rpm×10 min). All experiments were conducted in triplicate and compared to reactions with inhibitor and substrate but without UDPGA. Possible interfering peaks in the HPLC traces were identified by analysis of metabolic incubations with and without UDPGA in the absence of substrate. Inhibitor efficacy was confirmed in repeated experiments.

Raloxifene Incubations with Recombinant UGT Enzymes

Raloxifene substrate (50 μM; 5 μl of a methanol stock solution) and inhibitor (5 μl of a methanol stock solution) or vehicle were pre-incubated with Supersomes® or Bacculosomes® containing recombinant human UGT enzymes (250, 500 or 1000 μg/ml microsomal protein) and 10 mM MgCl₂ in 100 mM Tris HCl buffer (pH 7.5) for 5 min at 37° C. Metabolic reactions were started by addition of UDPGA to give a final UDPGA concentration of 1 mM and a total reaction volume of 500 μl. Reactions were stopped after 15 min at 37° C., then extracted and analyzed as described above.

Substrate Dependence of Metabolism

The substrate dependence of raloxifene metabolism was measured using UGT1A1, UGT1A3, UGT1A9 Supersomes® (250 μg/ml) and UGT1A10 Bacculosomes® (500 μg/ml). Raloxifene metabolism was measured over a 20-fold concentration range (5, 10, 20, 50, 100 μM) using the standardized incubation conditions described above in raloxifene incubations with recombinant UGT enzymes. Experiments were conducted in duplicate.

Analysis of Incubation Mixtures

Raloxifene and its two glucuronidation products were separated on a Rainin Microsorb-MV™ C-4 analytical column (5 μm; 4.6×250 mm). Compounds were eluted using a binary solvent gradient system. Solvent A was water brought to pH 8 with NH₄OH. Solvent B was methanol. Solvent flow rate was 1 ml/min and the column temperature was maintained at 50° C. Detection was at 280 nm. The initial mobile phase was 80% A and 20% B. Immediately upon sample injection, the concentration of B was increased linearly over 8 min to a final concentration of 85% B which was maintained for 4 min. The system was then returned to the initial conditions and equilibrated for 3 min. Retention times were 7.2 min and 7.8 min for glucuronide products G1 and G2 respectively, 9.9 min for nifedipine, 10.6 min for diethylstilbestrol and 12.3 min for raloxifene.

LC-MS analysis of microsomal incubation mixtures utilized a Rainin Microsorb-MV™ C-8 analytical column (5 μm; 4.6 mm×150 mm). Compounds were eluted using a binary solvent gradient where solvent A was water brought to pH 8 with NH₄OH and solvent B was 50:50 acetonitrile-methanol. The initial mobile phase was 80% A and 20% B. Immediately upon sample injection, the concentration of B was increased linearly over 10 min to a final concentration of 80% B which was maintained for 4 min. Solvent flow rate was 0.5 ml/min, column temperature was 50° C. Retention times were 9.1 min and 9.7 min for glucuronides G1 and G2 respectively, and 15.8 min for raloxifene. Raloxifene and its two glucuronides were analyzed by ESI mass spectrometry using scan ion monitoring. The mass spectrometer was run in the positive ion mode with N₂ drying gas flow of 12 L/min, drying gas temperature 350° C., nebulizer pressure 50 psig, chamber current 0.59 μA, capillary current 31 nA and capillary voltage (V_{cap}) 4000 V.

Quantitation of Raloxifene Glucuronides

Approximate standard curves for raloxifene glucuronides G1 and G2 were generated by comparing glucuronide formation to raloxifene loss from incubation mixtures assuming raloxifene glucuronidation was the only metabolic pathway. Raloxifene (1, 2, 5, 10, 20, 50 μM) was incubated with human liver microsomes (250 μg/ml) or Gentest UGT1A1 Supersomes® 250 μg/ml) and 1 mM UDPGA for 60 min. Residual raloxifene concentrations were calculated from raloxifene standard curves (1, 2, 5, 10, 20, 50 μM). Experiments were conducted in triplicate and compared to duplicate samples where UDPGA was omitted. Standard curves generated for raloxifene were linear over the concentration range tested (r²≧0.99). Standard curves generated from metabolism in liver microsomes and UGT1A1 Supersomes® were identical to those generated with liver microsomes.

β-Glucuronidase Cleavage of Raloxifene Glucuronides

Raloxifene substrate (50 μM; 10 μl of an acetonitrile stock solution) was pre-incubated with 250 gg/ml human liver microsomal protein (lot 083000) and 10 mM MgCl₂ in 100 mM Tris HCl buffer (pH 7.5) for 5 min at 37° C. Metabolic reactions were started by addition of UDPGA to give a final UDPGA concentration of 1 mM and a total reaction volume of 5 ml. After 15 min at 37° C., 500 μl of the incubation mixture was removed and extracted and analyzed as described above. The remaining incubation mixture was divided into two equal 2 ml samples, one of which was added to 2 ml of 100 mM sodium acetate buffer (pH 4.5) while the other was added to 2 ml 100 mM sodium acetate buffer (pH 4.5) containing 1000 units/ml of β-glucuronidase enzyme (Sekikawa, et al. 1995 Apparent intramolecular acyl migration and hydrolysis of furosemide glucuronide in aqueous solution. Biol. Pharm. Bull. 18: 134-9). The mixtures were incubated at 37° C. and 500 μl samples were taken at 5, 10, 20, 30, 40, 60 and 120 min. Samples were extracted as described above. All experiments were conducted in triplicate. Just prior to HPLC analysis, all samples were basified by addition of 5 μl of NH₄OH. Failure to do this resulted in poor peak shape and inadequate metabolite resolution in the HPLC trace.

Results—Identification of Raloxifene Glucuronidation Products

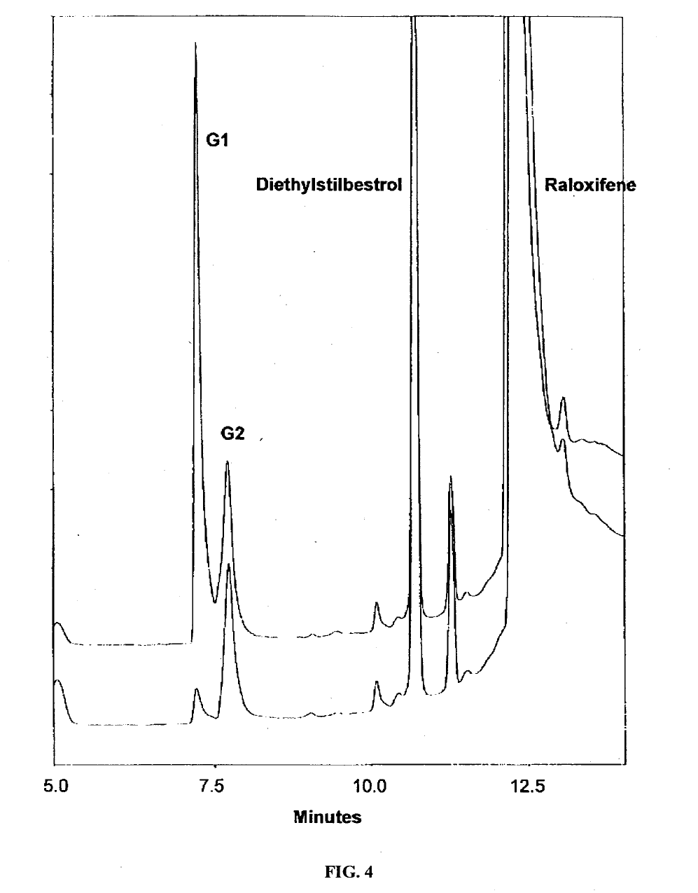

Microsomal metabolism of raloxifene resulted in two UDPGA-dependent metabolite peaks with retention times of 7.2 (G1) and 7.8 min (G2) (FIG. 4). LC-MS analysis demonstrated that the molecular weights of G1 and G2 (MH⁺m/z=650.3) were 176 units higher than raloxifene (MH⁺ m/z=474.1), consistent with addition of glucuronic acid. Additional confirmation that the raloxifene metabolites G1 and G2 were glucuronide adducts was achieved by incubation of glucuronide-containing microsomal incubation mixtures with β-glucuronidase enzyme. In the presence of β-glucuronidase, both G1 and G2 were completely removed from the incubation mixtures within 5 min. G1 and G2 were stable for at least 2 hr in acetate buffer (pH 4.5) in the absence of β-glucuronidase.

Results—Metabolic Incubation Conditions

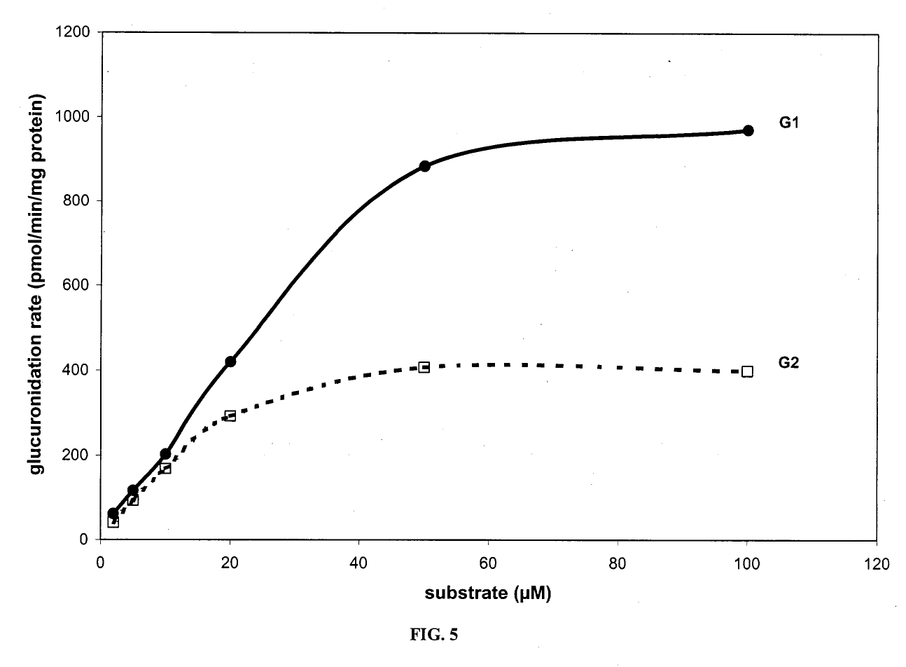

Raloxifene has limited solubility in aqueous media such that the maximum raloxifene concentration achieved in microsomal incubation mixtures was 100 μM. Formation of G1 and G2 was linear for at least 20 min at all substrate concentrations tested. Metabolism was linear with respect to microsomal protein concentration from 100-500 μg/ml (higher concentrations were not tested). Standardized incubations utilized 250 μg/ml microsomal protein, 1 mM UDPGA and a 15 min incubation time. Under these conditions, formation of G1 and G2 appeared to be saturated at a substrate concentration between 50 and 100 μM (FIG. 5). Whether this is true saturation or a function of limiting solubility could not be determined. At at a 50 μM substrate concentration the formation rates of G1 in human liver microsomes (mean i sd) were 896±15 (lot 083000), 960±51 (lot 032101) and 444±32 (lot 062101) pmol/min/mg. Formation rates for G2 were 557±7 (lot 083000) 610±19 (lot 032101) and 570±39 (lot 062101) pmol/min/mg. Eadie-Hofstee plots suggested that at least 3 enzymes were involved in raloxifene glucuronidation by human liver microsomes.

Raloxifene glucuronidation was also evaluated using pooled human jejunum microsomes. Small intestinal microsomes demonstrated greater metabolic activity than hepatic microsomes and favored formation of G2 over G1. Standardized incubations utilized 100 Pig/ml microsomal protein, 1 mM UDPGA and a 30 min incubation time. Under these conditions, the formation rates of G1 and G2 were 637±35 and 2,224±105 pmol/min/mg respectively.

Results—Raloxifene Glucuronidation by UGT Enzymes

Raloxifene glucuronidation was measured in insect microsomes expressing recombinant UGT1A1, 1A3, 1A4, 1A6, 1A7, 1A8, 1A9, 1A10, 2B7 and 2B15. Significant glucuronidation was only observed for UGT1A1, 1A3, 1A8, 1A9 and 1A10 (Table 6). UGT1A7 metabolized raloxifene only sparingly. No formation of G1 or G2 was observed for UGT1A4, 1A6, 2B7, or 2B15 at protein concentrations up to 1 mg/ml. No metabolism was observed in untransfected microsomes. G1 was the major metabolite formed for UGT1A1, 1A3 and 1A9. G2 was the predominant metabolite formed by UGT1A8 and UGT1A10. Both metabolites were formed equally by UGT1A7. As observed for 7-HFC metabolism, raloxifene glucuronidation was significantly lower in Bacculosomes® compared to Supersomes®.

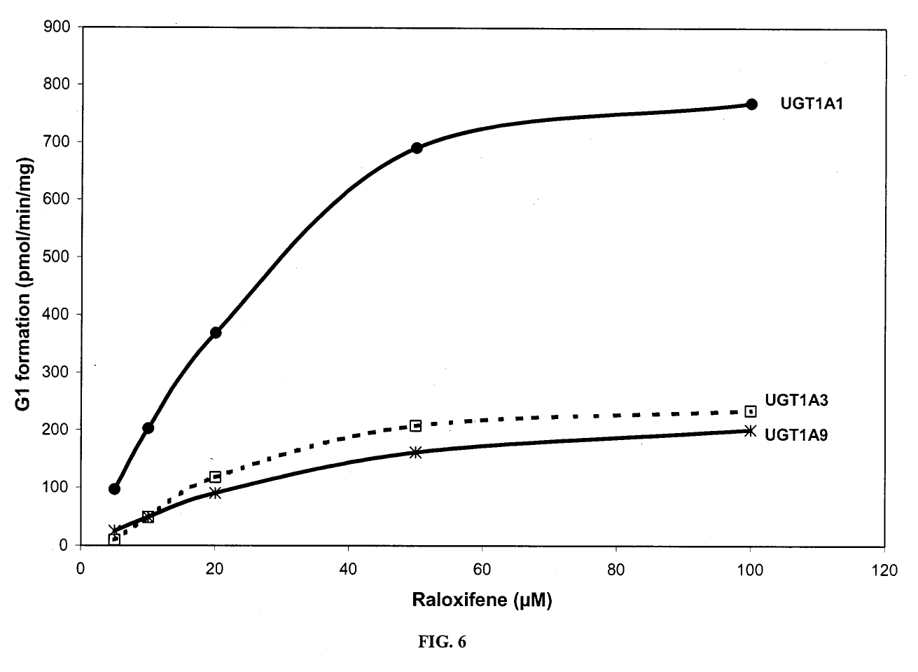

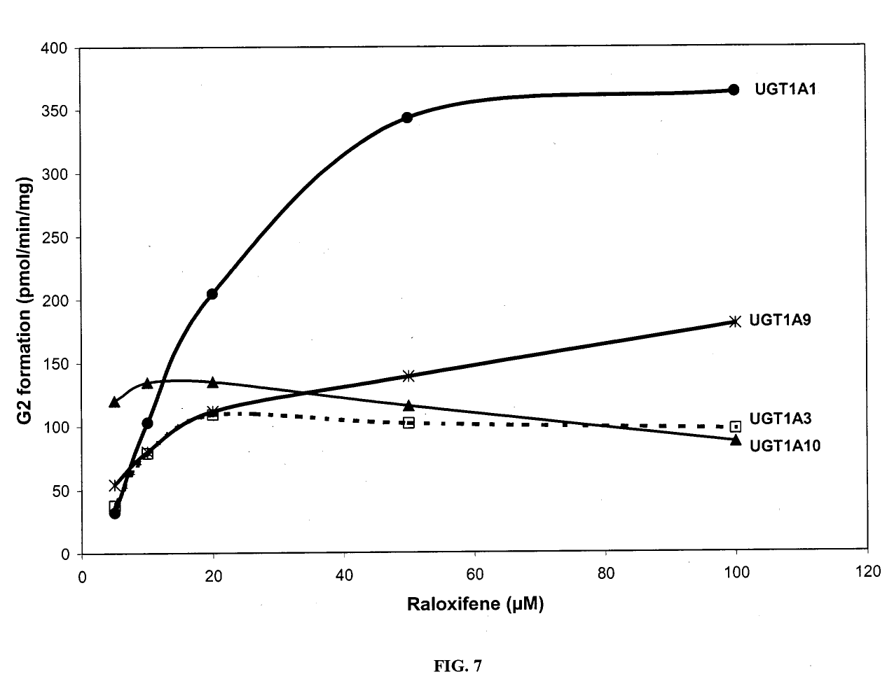

The substrate-dependence of raloxifene metabolism was evaluated with UGT1A1, 1A3 and 1A9 Supersomes® as well as UGT1A10 Bacculosomes® (FIGS. 6 and 7). Plots of metabolism rate versus substrate concentration indicated that the raloxifene glucuronidation rate reached its maximum between 50 and 100 μM for UGT1A1, 1A3 and 1A9 and between 10 and 20 μM for UGT1A10. Some caution must be exercised in evaluating this data, however, as the apparent saturation of metabolism under the conditions used may be a function of limiting substrate solubility (100 μM was the maximum raloxifene concentration achievable in microsomal incubation mixtures).

Results—Inhibition of Raloxifene Metabolism in Human Liver Microsomes and Intestinal Microsomes

Data comparing the inhibition of raloxifene microsomal metabolism by the UGT inhibitors of the present invention with inhibition by known UGT substrates or inhibitors are presented in Tables 7 and 8. Complete data for drugs, endogenous substrates, GRAS compounds and food additives tested are included in Table A3. Tannic acid and quercetin were excellent inhibitors of UGT-mediated raloxifene metabolism. Eugenol, silibinin, octyl gallate and the green tea compounds epicatechin gallate and epigallocatechin gallate were also good inhibitors of raloxifene metabolism in human liver and jejunum microsomes. It should be noted that the effects of these compounds varied somewhat between liver microsome lots, consistent with varying levels of UGT enzyme expression. Eugenol and silibinin are the major components of clove oil and silymarin (from Silybum marianum) respectively. Clove oil, silymarin and benzoin powder were good inhibitors of raloxifene metabolism, with IC₅₀ values between 5 and 10 μg/ml (Table 7). The UGT inhibitor compounds of the present invention were all better inhibitors than typical UGT substrates/inhibitors such as 17-α-ethinylestradiol, diflunisal and 4-methylumbelliferone. Unexpectedly, a large number of known UGT substrates, including many of the NSAIDs, diuretics, and sex hormones were not particularly good inhibitors of raloxifene glucuronidation. Many of these substrates exhibited less than 25% inhibition of raloxifene glucuronidation even at the highest tested concentration (500 μM).

Results—UGT1A1, UGT1A3, UGT1A9. UGT1A10

Data comparing the effect of UGT inhibitors on raloxifene metabolism by UGT1A1, 1A3, 1A8 and 1A9 Supersomes® as well as UGT1A10 Bacculosomes® are presented in Table 9 and Table A4. Tannic acid was the best inhibitor examined, significantly reducing raloxifene metabolism by 4 of the tested UGT enzyme forms. The green tea component epigallocatechin gallate also demonstrated broad UGT inhibitory activity against. Quercetin was a good inhibitor of UGT1A1, 1A3 and 1A9, but exerted only a modest effect on UGT1A10 and was ineffective versus UGT1A8. Octyl gallate was an excellent inhibitor of UGT1A1 and 1A3, but was a very poor inhibitor of UGT1A9 and 1A10. Alternatively, eugenol and clovebud oil were ineffective as inhibitors of UGT1A1, 1A3, 1A8 and 1A10, but were amongst the best inhibitors of UGT1A9. Diclofenac and mycophenolic were poor inhibitors of all the enzyme forms studied. This was expected for diclofenac, which is primarily a substrate for UGT2B7, however mycophenolic acid is known to be metabolized by both UGT1A9 and 1A10 (Table 2) and was expected to inhibit raloxifene glucuronidation by these enzymes. The lack of a mycophenolic acid effect suggests this compound has a significantly lower affinity than raloxifene for UGT1A9 and UGT1A10.

### Example 3

### Metabolism studies with Zidovudine (AZT)

Zidovudine Incubations with Human Liver and Intestinal Microsomes

Zidovudine substrate (5 μl of a water stock solution) and inhibitor or vehicle (5 μl methanol) were pre-incubated with human liver microsomes (lot 062101; 1 mg/ml) or pooled jejunum microsomes (1 mg/ml) and 10 mM MgCl₂ in 100 mM Tris HCl buffer (pH 7.5) for 5 min at 37° C. Metabolic reactions were started by addition of UDPGA to give a final UDPGA concentration of 5 mM and a total reaction volume of 500 μl. Reactions were stopped after the required time by addition of 200 μl stop solution (94:6 methanol-glacial acetic acid). Samples were vigorously vortex mixed, then protein was precipitated by centrifugation (3000 rpm×10 min). All experiments were conducted in triplicate and compared to reactions with inhibitor and substrate but without UDPGA. Possible interfering peaks in the HPLC traces were identified by analysis of metabolic incubations with and without UDPGA in the absence of substrate. Inhibitor efficacy was confirmed in repeated experiments.

Zidovudine-5′-glucuronide was quantitated by comparison to standard curves prepared using authentic metabolite. Stock solutions of metabolite were incubated with human liver microsomes in the absence of UDPGA as described above for AZT. Standard curves were linear over the range tested (0.5-50 μM) with r²≧0.99.

Analysis of Zidovudine Incubation Mixtures

Zidovudine and its glucuronidation product were separated on a Rainin Microsorb-MV™ C-18 analytical column (5 μm; 4.6×250 mm). Compounds were eluted using a binary solvent gradient system where solvent A was dilute phosphoric acid (pH 3) and solvent B was acetonitrile. Solvent flow rate was 1.0 ml/min and the column temperature was maintained at 50° C. Detection was at 266 nm. The initial mobile phase was 95% A and 5% B. Immediately upon sample injection, the concentration of B was increased linearly over 15 min to a final concentration of 50% B at which time the system was returned to the initial conditions. Retention times were 8.3 min for AZT-glucuronide and 10.0 min for AZT.

LC-MS analysis of microsomal incubation mixtures utilized an HPLC method similar to that described above. Compounds were separated using a Rainin Microsorb-MV™ C-18 (5 μm, 4.6×150 mm) analytical column. Elution used a binary solvent gradient system where solvent A was dilute 100 mM sodium formate (pH 3) and solvent B was acetonitrile. Solvent flow rate was 1.0 mmin and the column temperature was maintained at 50° C. Detection was at 280 nm. The initial mobile phase was 80% A and 20% B. Immediately upon sample injection, the concentration of B was increased linearly over 10 min to a final concentration of 90% B which was maintained for 2 min. Sodium adducts of AZT and its glucuronide were analyzed by ESI mass spectrometry using scan ion monitoring. The mass spectrometer was run in the positive ion mode with N₂ drying gas flow of 12 L/min, drying gas temperature 350° C., nebulizer pressure 50 psig, chamber current 0.59 liA, capillary current 31 nA and capillary voltage (Vcap) 4000 V.

Results—Zidovudine Glucuronidation

Metabolism of zidovudine by human liver and jejunum microsomes resulted in one UDPGA-dependent peak in the HPLC trace with a retention time of 8.3 min. The metabolite co-eluted with authentic zidovudine-5′-glucuronide, and LC-MS analysis demonstrated that the molecular weight of the metabolite (M−3Na⁺ m/z=510.0) was 176 units higher than zidovudine, consistent with addition of glucuronic acid.

Zidovudine had limited solubility in stock solvents (buffer, water, methanol, acetonitrile), so the maximum substrate concentration tested in metabolic incubations with human liver microsomes was 500,M. Using 1 mg/ml liver microsomal protein and a large excess of UDPGA (5 mM), formation of zidovudine-5′-glucuronide was linear for at least 60 min, and glucuronidation rate was linear with respect to substrate concentration over the range tested (20-500 μM). Standardized incubations utilized 500 μM zidovudine substrate, 1 mg/ml microsomal protein, 5 mM UDPGA and a 30 min incubation time. Under these conditions, the formation rate for AZT was 942±30 pmol/min/mg protein. Metabolism was not saturated at this substrate concentration, which is significantly lower than the published Km for zidovudine glucuronidation by liver microsomes (3-5 mM) (Pacifici, et al. 1996). Zidovudine glucuronidation in human liver: interindividual variability. Int. J. Clin. Pharmacol. Ther. 34: 329-34).

Metabolism of zidovudine by pooled human jejunum microsomes was significantly lower than observed for liver microsomes. Negligible levels of zidovudine-5′-glucuronide were detected after 30 min using microsomal protein concentrations of 50, 100 and 250 μg/ml (5 mM UDPGA). Increasing the microsomal protein concentration to 1 mg/ml and extending the incubation time to 60 min provided meaningful metabolite levels for inhibition studies. Under these conditions, the formation rate for zidovudine-5′-glucuronide was 53±1 pmol/min/mg microsomal protein.

Results—Inhibition of Zidovudine Glucuronidation

Data comparing the inhibition of AZT metabolism in human liver microsomes by various compounds are presented in Table 10. The best inhibitors were also evaluated in pooled human jejunum microsomes (Table 11). Zidovudine is glucuronidated primarily, if not exclusively, by UGT2B7 (Barbier, et al. 1999 UGT2B23, a novel uridine diphosphate-glucuronosyltransferase enzyme expressed in steriod target tissues that conjugates androgen and estrogen metabolites. Endocrinology 140: 5538-48), which is present in the liver and the intestine (Table 1). In the current study, zidovudine metabolism was significantly inhibited by the UGT2B7 substrates diclofenac, estradiol and 17-α-ethinylestradiol. Other compounds causing significant inhibition were gallocatechin gallate, tannic acid, clovebud oil, menthol, peppermint oil, geraniol, capsaicin and capsaicin analogs. In contrast to the data for raloxifene, quercetin was ineffective as an inhibitor of zidovudine metabolism.

### Example 4

### Metabolism studies with Labetalol

Labetalol Metabolism

Labetalol substrate (5 μl of a methanol stock solution) and inhibitor or vehicle (5 μl) were pre-incubated with human liver microsomes (lot 121301), jejunum microsomes or UGT Supersomes® (500 μg protein/ml) and 10 mM MgCl₂ in 100 mM Tris HCl buffer (pH 7.5) for 5 min at 37° C. Metabolic reactions were started by addition of UDPGA to give a final UDPGA concentration of 2.5 mM and a total reaction volume of 500 μl. Reactions were stopped after 30 min at 37° C. by addition of 200 μl stop solution (82:2:16 acetonitrile-tetrahydrofuran-(NH₄)₂HPO₄ 50 mM) containing salicylamide (50 μM) as internal standard. Samples were vigorously vortex mixed then protein was precipitated by centrifugation (3000 rpm×10 min). Supernatants were analyzed by HPLC with fluorescence detection. All experiments were conducted in triplicate and compared to reactions without UDPGA. Possible interfering peaks in the HPLC traces were identified by analysis of metabolic incubations with and without UDPGA in the absence of substrate.

Analysis of Labetalol Metabolic Incubations

Labetalol and its glucuronidation products were separated on a Hamilton RPR-1 analytical column (5 μm; 4.1×150 mm). Compounds were eluted using a binary solvent gradient system where solvent A was 5:5:90 tetrahydrofuran-acetonitrile-(NH₄)₂HPO₄ 50 mM, while solvent B was 25:25:50 tetrahydrofuran-acetonitrile-(NH₄)₂HPO₄ 50 mM. Solvent flow rate was 0.75 ml/min and the column temperature was ambient. The initial mobile phase was 10% A and 80% B. Two min after sample injection, the concentration of B was increased linearly over 10 min to a final concentration of 80% B which was maintained for 5 min. The system was then returned to the initial conditions and equilibrated for 3 min prior to the next run. Labetalol and metabolites were measured by fluorescence detection using a Jasco model FP920 detector. Excitation and emission wavelengths were 370 nm 418 nm respectively. Retention times for salicylamide internal standard and labetalol were 6.6 min and 15.0 min respectively.

Results—Metabolism by Human Liver and Small Intestinal Microsomes

Microsomal metabolism of labetalol resulted in two predominant UDPGA-dependent metabolite peaks in the HPLC trace with retention times of 8.5 min (LG1) and 10.3 min (LG2). A third peak was also observed at 11.9 min (LG3), however the appearance of this peak was inconsistent and it could not be measured at low substrate concentrations. A comparison of relative labetalol metabolite levels in human liver and jejunum microsomes as well as UGT Supersomes® is presented in Table 13. Liver and jejunum microsomes demonstrated similar metabolic activity, preferentially forming LG1. Of the UGT enzyme forms tested, labetalol was only metabolized by UGT1A9 and UGT2B7. LG1 was the primary metabolite formed by UGT2B7, however UGT1A9 formed equivalent levels of both LG1 and LG2. No detectable metabolism was observed using UGT1A1, 1A3, 1A4, 1A6, 1A8, 1A10 or 2B15.

Results—Inhibition of Labetalol Metabolism

Data describing inhibition of labetalol metabolism in human liver microsomes are presented in Table 14. Formation of both LG1 and LG2 was effectively inhibited by diethylstilbestrol, quercetin, tannic acid, epigallocatechin gallate and clovebud oil. Diflunisal and peppermint oil inhibited labetalol glucuronidation at higher inhibitor concentrations.

### Example 5

### Metabolism Studies with Estradiol (E2)

Estradiol Metabolism in Hepatic and Jejunum Microsomes

E2 substrate (5 μl of an acetonitrile stock solution) was pre-incubated with human liver microsomes (250 μg protein/ml) or jejunal microsomes (50, 100, 250 tg/ml) and 10 mM MgCl₂ in 100 mM Tris HCl buffer (pH 7.5) for 5 min at 37° C. Metabolic reactions were started by addition of UDPGA to give a final UDPGA concentration of 5 mM and a total reaction volume of 500 ill. Reactions were stopped after the desired time at 37° C. by addition of 200 jil stop solution (94% acetonitrile 6% glacial acetic acid) containing 17-α-ethinylestradiol (50 μM) as internal standard. Samples were vigorously vortex mixed then protein was precipitated by centrifugation (3000 rpm×10 min). Supernatants were analyzed by HPLC with UV detection. All experiments were conducted in triplicate and compared to reactions without UDPGA. Possible interfering peaks in the HPLC traces were identified by analysis of metabolic incubations with and without UDPGA in the absence of substrate.

Estradiol Metabolism by Recombinant UGT Enzymes

E2 (50 μM) was incubated with UGT Supersomes® (250 μg/ml protein) or Bacculosomes® (500 μg/ml protein) for 30 min using conditions identical to those described above for human liver microsomes.

Analysis of Estradiol Metabolic Incubations

E2 and its glucuronidation products were separated on a Rainin Microsorb-MV™ C-18 analytical column (5 μm; 4.6×250 mm). Compounds were eluted using a binary solvent gradient system where solvent A was dilute phosphoric acid (pH 3) and solvent B was 80:20 acetonitrile-methanol. Solvent flow rate was 1.0 ml/min and the column temperature was maintained at 50° C. Detection was at 280 nm. The initial mobile phase was 80% A and 20% B. Immediately upon sample injection the concentration of B was increased linearly over 10 min to a final concentration of 90% B which was maintained for 2 min. The system was then returned to the initial conditions and equilibrated for 3 min prior to the next run. Retention times for the analytes were E2-3-(β-D-glucuronide) 8.0 min; E2-17-(β-D-glucuronide) 8.5 min; diethylstilbestrol glucuronide 8.75 min; E2 10.9 min; 17-α-ethinylestradiol 11.2 min; and diethylstilbestrol 11.55 min.

LC-MS analysis of microsomal incubation mixtures utilized an HPLC method similar to that described above except that solvent A was 100 mM sodium formate (pH 3) and solvent B was acetonitrile. Compounds were separated using a Rainin Microsorb-MV™ C-18 (5 μm, 4.6×150 mm) analytical column. Sodium adducts of E2, diethylstilbestrol and their glucuronides were analyzed by ESI mass spectrometry using scan ion monitoring. The mass spectrometer was run in the positive ion mode with N₂ drying gas flow of 12 L/min, drying gas temperature 350° C., nebulizer pressure 50 psig, chamber current 0.59 μA, capillary current 31 nA and capillary voltage (V_{cap}) 4000 V.

Quantitation of Estradiol Glucuronides

Standard curves of E2 were prepared by incubation of stock solutions (1, 2, 5, 10, 20, 50 gM) with human liver microsomes for 5 min in the absence of UDPGA as described above. The identical procedure was employed to generate standard curves of E2-3-β-D-glucuronide and E2-17-β-D-glucuronide (0.1, 0.2, 0.5, 1, 2, 5, 10, 20 μM). Standard curves (normalized HPLC peak area versus concentration) were linear over the concentration range tested (r²≧0.99). Standard curves for E2 and E2-17-(β-D-glucuronide) were superimposable, however normalized HPLC peak areas for E2-3-(β-D-glucuronide) were only half those for equivalent concentrations of E2-17-(β-D-glucuronide). This difference appears to be due to a 50% lower UV extinction coefficient for the 3-β-D-glucuronide.

β-Glucuronidase Assays

E2 (50 liM) was metabolized by human liver microsomes as described above using a total reaction volume of 4 ml. After 30 min at 37° C., 500 gl of the incubation mixture was removed, extracted and analyzed as above. The remaining incubation mixture was divided into two equal 1.5 ml samples, one of which was added to 1.5 ml of 100 mM sodium acetate buffer (pH 4.5) while the other was added to 1.5 ml 100 mM sodium acetate buffer (pH 4.5) containing 1000 units/ml of β-glucuronidase enzyme. The mixtures were incubated at 37° C. and 500 μl aliquots were taken at 0.25, 0.5, 1, 2, and 3 h. Samples were extracted and analyzed as described above. All experiments were conducted in triplicate.

Inhibition of Estradiol Metabolism

E2 substrate (5 μl of an acetonitrile stock solution) and inhibitor or vehicle (5 μl methanol) were pre-incubated with human liver (250 μg protein/ml) or jejunum microsomes (50 μg/ml) and 10 mM MgCl₂ in 100 mM Tris HCl buffer (pH 7.5) for 5 min at 37° C. Metabolic reactions were started by addition of UDPGA to give a final UDPGA concentration of 5 mM and a total reaction volume of 500 pi. Reactions were stopped after 30 min at 37° C. by addition of 200 μl stop solution then extracted and analyzed as described above.

Results—Characterization of E2 Metabolites

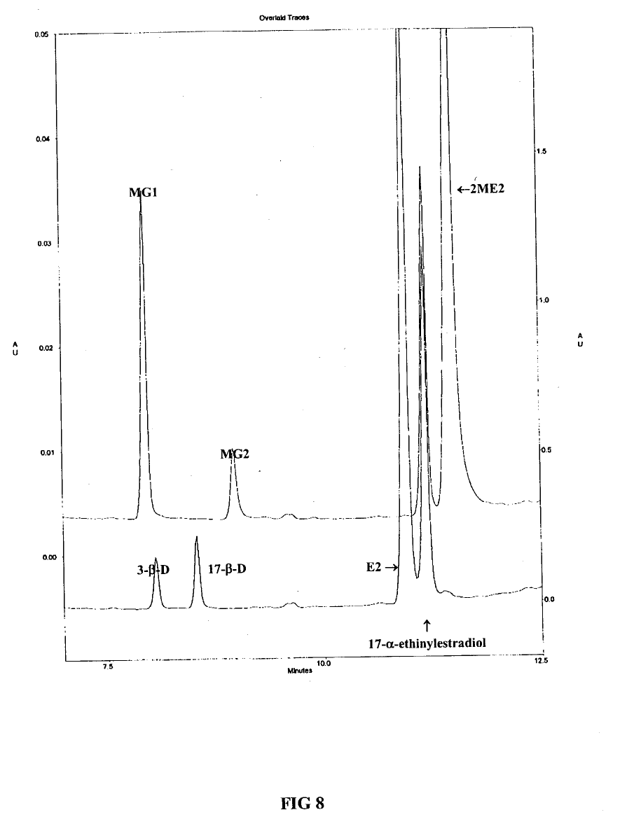

Microsomal metabolism of E2 resulted in two UDPGA-dependent metabolite peaks in the HPLC trace. The peak at 8.0 min co-eluted with authentic samples of E2-3-β-D-glucuronide while the peak at 8.5 min co-eluted with E2-17-β-D-glucuronide (FIG. 8). LC-MS analysis demonstrated that the molecular weight of both metabolites (M−2Na⁺ m/z=493.4) was 176 units higher than E2, consistent with addition of glucuronic acid. Further confirmation that the metabolites were glucuronide adducts was achieved by incubation of glucuronide-containing microsomal incubation mixtures with β-glucuronidase enzyme. In the presence of β-glucuronidase, both 3-(β-D-glucuronide) and 17-(β-D-glucuronide) were completely removed from the incubation mixture within 15 min.

Results—Metabolism by Human Liver and Small Intestinal Microsomes

The time-course of E2 metabolism by human liver microsomes was measured for 60 min over a 500-fold concentration range (2-1000 μM). Using a microsomal protein concentration of 250 μg/ml and an excess of UDPGA (5 mM), formation of E2-3-μ-D-glucuronide, E2-17-β-D-glucuronide, and corresponding loss of E2, were linear for 60 min. Metabolism by human liver microsomes appeared saturated at a 100 μM estradiol substrate concentration and no substrate-mediated inhibition was observed at estradiol concentrations up to 500 μM.

A comparison of E2 metabolism rates at 5 and 50 μM substrate concentrations is presented in Table 12. Metabolism of E2 by liver microsomes was moderate compared to other UGT substrates evaluated in similar screens. The total loss of E2 from liver microsomal incubation mixtures after 60 min was 56% when 5 μM substrate was used and 31% for a 50 ptM substrate concentration. Mass balance calculations for E2 metabolism indicate that formation of the two glucuronides accounted for all of the E2 lost during the incubation (Table 12). Metabolism of E2 by pooled human jejunal microsomes was 9-to 10-fold higher than observed for the two lots of liver microsomes, resulting in almost exclusive formation of E2-3-(β-D-glucuronide).

Results—Inhibition ofE2 Metabolism

Data describing inhibition of E2 metabolism in human liver and jejunum microsomes are presented in Table 15. Formation of E2-3-(β-D-glucuronide) was inhibited by quercetin, tannic acid, epicatechin gallate, epigallocatechin gallate, gallocatechin gallate and silymarin, however formation of E2-17-(β-D-glucuronide) was minimally impacted by these compounds. Formation of E2-17-(β-D-glucuronide) was preferentially inhibited by allspice berry, clovebud oil and peppermint oil. Diethylstilbestrol was a weak inhibitor of E2-3-(β-D-glucuronide) formation in liver microsomes, but activated formation of E2-17-(β-D-glucuronide). This activation was not observed in human jejunum microsomes.

### EXAMPLE 6

### Metabolism Studies with 2-Methoxyestradiol (2ME2)

2ME2 Metabolism by Hepatic and Jeiunal Microsomes

2ME2 was incubated with human liver (250 μg/ml protein) and jejunum microsomes (50, 100, 250 μg/ml), UGT Supersomes® (250 μg/ml protein) and Bacculosomes® (500 μg/ml protein) using conditions identical to those described above for E2.

Results—Characterization of 2ME2 Metabolites

Microsomal metabolism of 2ME2 resulted in two UDPGA-dependent metabolite peaks with retention times of 7.9 min (MG1, the major peak) and 8.95 min (MG2, the minor peak) (FIG. 8). LC-MS analysis demonstrated that the molecular weight of both metabolites (M−Na⁺ m/z=501.5; M−2Na⁺=523.3) was 176 units higher than 2ME2 (M−Na⁺ m/z=325.3), consistent with addition of glucuronic acid. Further confirmation that the metabolites were glucuronide adducts was achieved by incubation of glucuronide-containing microsomal incubation mixtures with β-glucuronidase enzyme. In the presence of β-glucuronidase, both MG1 and MG2 were completely removed from the incubation mixture within 15 mm. The chemical structure of the 2ME2 metabolites could not be conclusively determined in these studies, however comparison of the 2ME2 metabolite profile with that observed for E2 suggests that the more polar metabolite (MG1) is 2-methoxyestradiol-3-(β-D-glucuronide) while the less polar product (MG2) is likely 2-methoxyestradiol-17-(β-D-glucuronide).

Results—Metabolism by Human Liver and Small Intestinal Microsomes

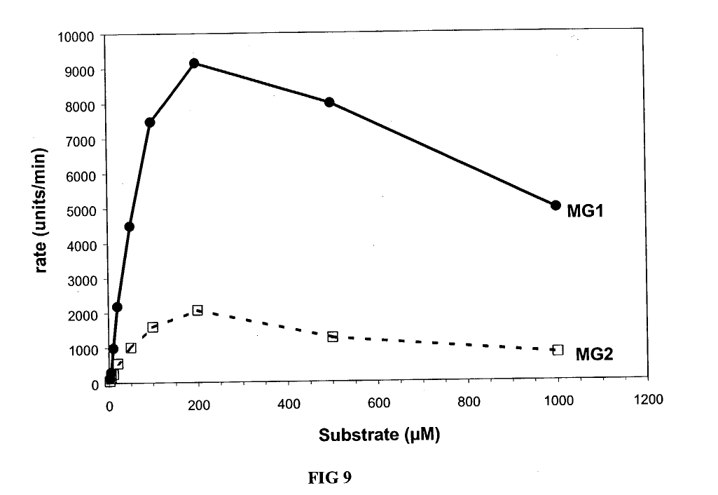

The time-course of 2ME2 metabolism by human liver microsomes was measured for 60 min over a 500-fold concentration range (2-1000 FM). Using a microsomal protein concentration of 250 μg/ml and an excess of UDPGA (5 mM), formation of MG1 and MG2, and corresponding loss of 2ME2, was linear for 30 min. A plot of glucuronide formation rate versus substrate concentration showed a reduction in glucuronide formation at substrate concentrations higher than 200 μM (FIG. 9). This does not arise from reduced solubility of the 2ME2 substrate at the highest concentrations, and suggests substrate-mediated inhibition of 2ME2 glucuronidation. Metabolism was also measured under conditions favoring interaction of 2ME2 with cytochrome(s) P450. Negligible NADPH-dependent metabolism was observed in human liver microsomes (data not shown) indicating that glucuronidation is the dominant route of 2ME2 metabolism.

A comparison of 2ME2 metabolism rates in human liver and jejunum microsomes is presented in Table 16. Metabolism of 2ME2 by liver microsomes was moderate compared to other UGT substrates evaluated in similar screens. The total loss of 2ME2 from liver microsomal incubation mixtures after 60 min was 42% for a 50 μM substrate concentration. In the absence of authentic metabolite standards, the absolute formation rates of MG1 and MG2 could not be determined. A comparison of normalized HPLC peak areas suggests that MG1 was preferred over MG2. Some caution must be exercised in making this comparison, however, as MG1 and MG2 may have different UV extinction coefficients. Metabolism of 2ME2 by pooled human jejunal microsomes was dramatically higher than observed for the two lots of liver microsomes, resulting in almost exclusive formation of MG1. As indicated in Table 16, 2ME2 loss was 20-times greater in jejunal microsomes than in the most active batch of liver microsomes (lot 032101).

Results—Metabolism by Recombinant UGT Enzymes

The contribution of different UGT enzymes to 2ME2 metabolism was evaluated using microsomes from insect cells transfected with human UGT enzyme forms (Supersomes® and Bacculosomes®). The absence of authentic standards precluded calculation of glucuronide levels in these incubations, so data are reported as metabolite peak areas relative to those observed in human liver microsomes (Table 17). Greatest metabolism was observed for UGT1A10, which formed MGl exclusively. The rate of 2ME2 (50 μM) loss from incubations with UGT10 Supersomes was 3056±43 pmol/min/mg (mean±SD). This rate is 2-fold higher than observed in human liver microsomes, but 10-fold lower than observed for small intestinal microsomes. UGT1A8 had similar activity to UGT1A10 and 2377+57 (pmol/min/mg), and also favored formation of MG1. UGT1A1, a major hepatic UGT form, was approximately half as active as UGT1A10 (1406±156 pmol/min/mg). UGT1A3 and 1A9 formed both MG1 and MG2, however both demonstrated less than 10% of the activity of UGT1A10. UGT1A4, UGT2B7 and UGT2B15 all formed MG2 as the exclusive metabolite, albeit at very low levels. No 2ME2 metabolism was observed using UGT1A6.

Results—Inhibition of 2ME2 Metabolism

Data describing inhibition of 2ME2 metabolism in human liver and jejunum microsomes are presented in Table 18. Formation of MG1 was inhibited by quercetin, tannic acid, epicatechin gallate, epigallocatechin gallate, gallocatechin gallate and raloxifene, however inhibition of MG2 formation required relatively high concentrations of these compounds. Formation MG2 was preferentially inhibited by diclofenac, 17-α-ethinylestradiol, naringenin, and peppermint oil. As observed for E2, diethylstilbestrol was a weak inhibitor of MG1 formation in liver microsomes, but activated formation of MG2. This activation was not observed in human jejunum microsomes

### EXAMPLE 7

### In Vivo Inhibition of Raloxifene Glucuronidation by Quercetin

In Humans

Raloxifene (Evista®; 60 mg) is administered to 12 healthy volunteers with water (150 ml) alone or with quercetin (500 mg tablet). Quercetin is widely marketed by vitamin and supplement companies as an antioxidant, and has been used at doses from 400-1500 mg/day without reported toxicities. Venous blood samples are collected prior to each dose (time 0) and at 0.5, 1, 1.5, 2, 3, 4, 6, 8, 12 and 24 h post-dose. Erythrocytes are precipitated using standard centrifugation techniques, and then plasma samples are analyzed for raloxifene and metabolites using a validated LC-MS method. Appropriate pharmacokinetic parameters (Cₘₐₓ, tₘₐₓ, AUC) are calculated using non-compartmental methods and data for the different doses are compared using an unpaired t-test. Quercetin is considered to be effective if it results in at least a 25% increase in raloxifene AUC or causes a significant reduction in the variability in raloxifene levels.

### EXAMPLE 8

### In Vivo Inhibition of Raloxifene Glucuronidation by Quercetin Pharmacokinetic Study in Female Rats

The effect of UGT inhibitors on raloxifene oral bioavailability was measured in female rats. In this study, separate groups of 6 female rats were administered raloxifene (10 mg/kg) by oral gavage alone and with the UGT inhibitors quercetin, tannic acid, or diflunisal (each 50 mg/kg). Plasma levels of raloxifene were measured over a 24 h time period and raloxifene pharmacokinetics compared in the presence and absence of the UGT inhibitor.

Female Sprague-Dawley rats (220-250 g body weight) with cannulae inserted into the jugular vein were purchased from Hilltop Animals Inc. (Scottsdale, Pa.). Catheter patency was maintained using a heparin lock. Animals were individually housed at 18-26° C. and allowed free movement and access to water. Rats were fed standard Laboratory Rodent Diet during a minimum 1-day acclimatization period but were fasted from at least 8 h prior to dose administration and were not administered food throughout the study. One rat treated with raloxifene and tannic acid was found dead 24 h after dose administration, however no other toxic signs were observed in any of the other animals throughout the study.

Dosing solutions were prepared as follows: raloxifene (10 mg/ml in ethanol; 2.5 ml) was vortex mixed with inhibitor (50 mg/ml in ethanol; 2.5 ml) and polyethylene glycol 400 (PEG 400; 2.5 ml). Immediately prior to treatment, the ethanol was removed by nitrogen evaporation and rats were administered 1 ml/kg of each emulsion using a standard gavage needle. Serial blood samples (500 μl) were drawn prior to the dose (time 0) and at 0.5, 1, 1.5, 2, 3, 4, 6, 8 and 24 h post-dose the jugular vein cannula. Blood volume was replaced with saline after each sample. Whole blood samples were collected in Microtainer® tubes (Becton-Dickinson, Franklin Lakes, N.J.) containing sodium EDTA anti-coagulant. Erythrocytes were precipitated by centrifugation (2800 rpm×10-20 min) and plasma samples were stored in the freezer prior to extraction and analysis.

Plasma samples (100 μl) were extracted by vortex mixing for 60 sec with 200 μl extraction solvent (80% acetonitrile 20% 2 mM NH₄OAc pH 9) and 20 μl internal standard solution (1 μM tamoxifen in methanol). Precipitated materials were separated by microcentrifugation (14000 rpm×5 min) then the supernatants were filtered into Eppendorf tubes and subjected to further cenrtigugation (4000 rpm×5 min). Supernatants were analyzed for raloxifene using a validated HPLC-MS method. Raloxifene plasma concentrations were quantified by comparison with standard curves generated from spiked plasma samples extracted in the same manner as the test samples. The lower limit of quantitation was 2 ng/ml.

Peak blood raloxifene concentrations (Cₚₑₐₖ) and time to achieve these concentrations (Tₚₑₐₖ) were measured directly from concentration vs time profiles. Area under the concentration vs time curve from 0-8 h (AUC₀₋₈) and 0-24 h (AUC₀₋₂₄) were calculated using the linear trapezoidal method. Raloxifene pharmacokinetics in the presence of UGT inhibitors were compared to those in the raloxifene-only control using an unpaired t-test (normally distributed data) or the Mann-Whitney rank-sum test.

Results—Pharmacokinetic Study in Female Rats

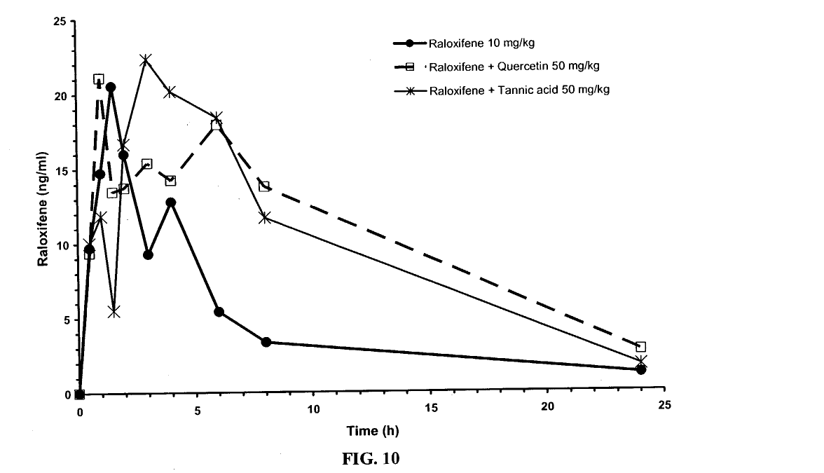

Raloxifene pharmacokinetics alone and in the presence of the UGT inhibitors quercetin, tannic acid and diflunisal are presented in Table 19. Mean concentration versus time profiles for raloxifene are presented in FIG. 10. Consistent with published reports, raloxifene pharmacokinetics were highly variable. Raloxifene is known to undergo significant enterohepatic recirculation and 2 raloxifene peak concentrations were observed in almost all animals. Diflunisal had no significant effect on raloxifene oral bioavailability, however quercetin and tannic acid both caused a statistically significant 2-fold increase in raloxifene AUC₀₋₂₄. These experiments suggest that UGT inhibitors such as quercetin may have clinical utility as a means of improving raloxifene oral bioavailability.

All publications and patent applications mentioned in this specification are herein incorporated, by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

TABLE 1
Tissue distribution of UGT enzymes in the gastrointestinal tract and kidney

| Tissue | 1A1 | 1A3 | 1A4 | 1A5 | 1A6 | 1A7 | 1A8 | 1A9 | 1A10 | 2B4 | 2B7 | 2B10 | 2B11 | 2B15 | 2B17 | 2B23 |
|---|---|---|---|---|---|---|---|---|---|---|---|---|---|---|---|---|
| Liver | ● | ● | ● | — | ● | — | — | ● | — | ● | ● | ● | ● | ● | ● | |
| Biliary epithelia | ● | ● | ● | — | ● | — | — | — | ● | | | | | | | |
| Esophagus | — | — | — | — | — | ● | ● | ● | ● | — | ● | ● | | ● | | |
| Stomach | ◯ | ◯ | — | — | ◯ | ◯ | — | ◯ | ● | | | | | | | |
| Duodenum | ◯ | ◯ | ◯ | — | ◯ | — | — | — | ● | ◯ | ◯ | — | | ◯ | | |
| Jejunum | ◯ | ● | ◯ | — | ◯ | — | ◯ | — | ● | ◯ | ◯ | — | | ● | | |
| Ileum | ◯ | ◯ | ◯ | — | ◯ | — | ◯ | — | ● | ◯ | ◯ | ◯ | | ● | | |
| Colon | ● | ● | ● | — | ● | — | ● | ● | ● | — | ● | | | | | |
| Kidney | — | | | | ● | | — | ● | — | — | ● | | ● | ● | ● | |
| Caco-2 cells | | □ | | | □ | | | □ | | | □ | | | | | |

● = present in all samples tested.
◯ = present in some of the samples tested.
□ = Expression dependent on the cell-line clone and passage number.
— = not present in any of the samples tested.

TABLE 2
Substrates for different UGT enzymes

| Compound | 1A1 | 1A3 | 1A4 | 1A6 | 1A7 | 1A8 | 1A9 | 1A10 | 2B4 | 2B7 | 2B15 | 2B17 |
|---|---|---|---|---|---|---|---|---|---|---|---|---|
| Acetaminophen | — | | — | ● | | — | ● | — | | — | — | |
| Amitriptyline | — | ◯ | ● | — | — | — | — | — | | | | |
| Androsterone | — | ● | ● | — | — | — | — | ◯ | | ● | — | ● |
| AZT (Zidovudine) | — | | | — | | | — | | — | ● | — | — |
| Bilirubin | ● | | ● | — | | | — | | | — | | — |
| Carvacrol | ◯ | | ● | ● | | ● | ● | — | | | ● | |
| Codeine | | — | — | | | — | | — | | ● | — | |
| Diclofenac | | | ● | | | | | | | ● | | |
| Diethylstilbestrol | — | | | | | ● | ● | — | | | — | |
| Diflunisal | | | — | | | ● | ● | — | | ● | | |
| 17-β-Estradiol | ● | ● | ● | — | — | — | ◯ | ● | | ● | — | — |
| 2-hydroxyestradiol | ● | ● | ● | | ● | ◯ | ● | ● | | ● | — | |
| Estriol | ◯ | | ● | — | ● | — | ◯ | ● | ● | ● | — | — |
| 2-hydroxyestriol | ● | | ● | | — | — | — | — | | ● | ● | |
| 4-hydroxyestradiol | ◯ | ● | ● | | | ● | ● | | | ● | ● | |
| Estrone | — | ● | — | | — | ◯ | ● | ◯ | | | — | — |
| 2-hydroxyestrone | ● | ● | — | | | ● | ● | ● | | ● | ● | |
| 4-hydroxyestrone | ◯ | ● | — | | — | ◯ | ● | ● | — | ● | ● | |
| 17-α- | ● | | ● | — | | ● | ● | — | | — | — | |
| Ethinylestradiol | | | | | | | | | | | | |
| Eugenol | ● | ● | ● | ● | | ● | ● | ● | ● | | ● | ● |
| Ibuprofen | — | ● | — | | — | — | ◯ | — | | ● | | |
| Imipramine | — | ● | ● | — | — | — | — | — | | | — | |
| Indomethacin | | | ● | | | | — | | | ● | | |
| Irinotecan SN-38 | ● | ● | ● | ● | ● | ● | ● | ● | | — | | |
| Kaempferol | | | | | | | ● | | | | | |
| Labetalol | — | | | | | | ● | | | | | |
| Linoleic acid | | | | | | | | | | ● | | |
| Menthol | — | — | ● | — | | — | — | | | ● | — | |
| 4- | ● | ● | — | | ● | ◯ | ● | ● | | ● | ● | — |
| Methyl- | | | | | | | | | | | | |
| umbelliferone | | | | | | | | | | | | |
| Morphine | — | ● | — | — | | ● | — | — | | ● | — | |
| Mycophenolic acid | — | — | — | — | | ● | ● | ● | — | — | | |
| Naproxen | ◯ | ● | — | | | — | ● | | | ● | | |
| Naringenin | ● | ● | — | | | ● | ● | ● | | | ● | |
| Octyl gallate | ● | | | ● | ● | — | ● | ● | | | | |
| Oxazepam | | | | | | | | | | ● | | |
| p-Nitrophenol | ● | ● | ● | | ● | ◯ | ● | ◯ | | ● | ● | ● |
| Probenecid | — | | | — | | — | — | — | | ● | | |
| Propyl gallate | ● | | | ● | ● | — | ● | ● | | | | |
| Quercetin | ● | ● | — | — | | ● | ● | ● | | | ● | |
| Retinoic acid, trans | — | | — | | — | ◯ | ◯ | — | | ● | | — |
| Testosterone | — | — | ● | | — | ◯ | — | — | | — | ● | ● |
| Di- | — | | ● | | | ◯ | | ● | | | ● | ● |
| hydroxytestosterone | | | | | | | | | | | | |
| Valproic acid | | ● | | | | ● | | — | | ● | | |
| Vanillin | | | | ● | ● | — | ● | ● | | | | |

● = substrate.
◯ = mixed data in the literature (probably due to differing enzyme expression systems and activities).
— = not a substrate.

TABLE 3
7-HFC (50 μM) glucuronidation by recombinant UGT
enzymes (250 μg/ml) from Gentest (Supersomes ®) and
Panvera (Bacculosomes ®).

| UGT | Supersomes^{a} | Bacculosomes^{a} |
|---|---|---|
| 1A1 | 816 ± 70; 929 ± 7 | 346 ± 12 |
| 1A3 | 952 ± 82 | 17 ± 1 |
| 1A4 | b | — |
| 1A6 | 4904 ± 368 | 1528 ± 34 |
| 1A7 | — | 564 ± 9 |
| 1A8 | 486 ± 5 | — |
| 1A9 | 3976 ± 41 | — |
| 1A10 | 460 ± 2 | 553 ± 40 |
| 2B7 | 972 ± 72 | 212 ± 13 |
| 2B15 | 976 ± 17 | — |

^{a}Data are mean ± SD pmol/min/mg protein. Metabolism rates in human liver and jejunum microsomes are 26,405 ± 272 and 6,829 ± 61 pmol/min/mg, respectively.
b Below the limit of detection.
— = enzyme not available.

TABLE 4
Inhibition of 7-HFC glucuronidation in human liver microsomes
by various compounds.

| | Percentage of control metabolism at indicated inhibitor concentration (μM)^{a} | | | | |
|---|---|---|---|---|---|
| Inhibitor | 500 | 200 | 100 | 50 | 25 |
| Ascorbyl palmitate | 11 (3), 17 (0) | 51 (1) | 64 (1) | | |
| Capsaicin | 39 (0) | 42 (1), 44 (1) | 53 (1), 57 (0) | 69 (1) | |
| Carvacrol | 31 (1) | 63 (0) | 79 (1) | | |
| Diclofenac | 30 (1), 30 (1) | 52 (0) | 64 (2) | | |
| Diethylstilbestrol | 0.1 (0) | 1 (0) | 11 (1) | 34 (2) | 57 (1) |
| Diflunisal | 37 (1), 38 (0) | 65 (3) | 77 (2) | | |
| Dihydrocapsaicin | 42 (2) | 52 (2) | 60 (3) | | |
| Epicatechin gallate | 10 (1) | 55 (3) | 67 (1) | | |
| Epigallocatechin gallate | 5 (1) | 37 (2) | 67 (1) | | |
| 17-α-Ethinylestradiol | 72 (1) | | | | |
| Gallocatechin gallate | 0 (0) | 25 (0) | 54 (1), 46 (2) | 68 (2) | 80 (2) |
| Lauryl gallate | 18 (6) | 22 (1) | 43 (2), 49 (1) | 76 (2) | 92 (3) |
| Linoleic acid | 33 (1) | 77 (4) | 86 (3) | | |
| 4-Methylumbelliferone | 45 (1), 47 (2) | 64 (1) | 78 (1) | | |
| Octyl gallate | 0.1 (0.1) | 6 (0) | 26 (1), 29 (1) | 65 (0) | 86 (1) |
| Quercetin | 52 (1) | 53 (1) | 58 (1) | | |
| Retinoic acid | 40 (2) | 84 (3) | 91 (2), 57 (2) | | |
| Retinol | 25 (1) | 39 (1) | 59 (1) | | |
| Tannic acid | 0 (0) | 0 (0) | 0 (0) | 2 (0) | 14 (1), 18 (0) |
| N-Vanillylnonanamide | 48 (1) | 63 (2) | 69 (1) | | |

^{a}Data are mean (SD) of triplicate determinations. A smaller number indicates greater inhibition.

TABLE 5
Kinetic studies evaluating inhibitors of 7-HFC glucuronidation
in human liver microsomes^{a}

| | Kₘ | Vₘₐₓ | Kᵢ | Inhibition |
|---|---|---|---|---|
| Inhibitor (μM) | μM | nmol/min/mg | μM | Type |
| Tannic acid (0, 5, 10) | 85 ± 7 | 57 ± 3 | 9.8 ± 0.5 | Non-competitive |
| Tannic acid (0, 25) | 85 ± 7 | 57 ± 3 | 5.4 ± 0.4 | Non-competitive |
| Diethylstilbestrol | 87 ± 7 | 64 ± 4 | 22 ± 1 | Mixed |
| (0, 5, 10, 25, 50) | | | | |
| Octyl gallate (0, 50, 100) | 81 ± 7 | 57 ± 3 | 40 ± 5 | Mixed |
| Octyl gallate (0, 200) | 81 ± 7 | 57 ± 3 | 16 ± 2 | Non-competitive |
| Gallocatechin gallate | 75 ± 7 | 54 ± 3 | 53 ± 5 | Mixed |
| (0, 50, 100) | | | | |
| Gallocatechin gallate (0, 200) | 75 ± 7 | 54 ± 3 | 40 ± 3 | Non-competitive |
| Epigallocatechin gallate | 75 ± 7 | 54 ± 3 | 164 ± 18 | Mixed |
| (0, 50, 100, 200) | | | | |
| Diclofenac (0, 100, 200, 500) | 87 ± 7 | 64 ± 4 | 188 ± 14 | Mixed |
| Diflunisal (0, 100, 200, 500) | 92 ± 9 | 62 ± 4 | 218 ± 11 | Competitive |

^{a}Data are mean ± S.E. of estimate (r² > 0.99) determined by non-linear regression analysis.

TABLE 6
Raloxifene (50 μM) metabolism by recombinant UGT
enzymes from Gentest (Supersomes ®; 250 μg/ml)
and Panvera (Bacculosomes ®; 500 μg/ml)^{a}

| | Supersomes ® | | Bacculosomes ® | |
|---|---|---|---|---|
| UGT | G1 | G2 | G1 | G2 |
| 1A1 | 824 ± 36 | 396 ± 30 | 303 ± 16 | 158 ± 8 |
| 1A3 | 258 ± 15 | 146 ± 17 | 4.7 ± 0.3 | 2.2 ± 0.5 |
| 1A7 | — | — | 17 ± 1 | 18 ± 2 |
| 1A8 | 207 ± 13 | 697 ± 9 | — | — |
| 1A9 | 201 ± 11 | 164 ± 13 | — | — |
| 1A10 | — | — | 24 ± 2 | 133 ± 11 |

^{a}Data are mean ± SD pmol/min/mg protein

TABLE 7
Inhibition of raloxifene (50 μM) glucuronidation in human liver microsomes by various
compounds.

| | % of control metabolism at indicated compound concentration^{a} | | | |
|---|---|---|---|---|
| Inhibitor | 100 μM | 50 μM | 25 μM | 10 μM |
| Diclofenac | 50 (3), 53 (3) | 65 (1), 67 (1) | — | — |
| Diethylstilbestrol | 8 (0), 43 (0) | 14 (1), 58 (2) | 36 (1), 100 (5) | 58 (3), 111 (7) |
| Diflunisal | 30 (4), 34 (1) | 43 (1), 45 (2) | 63 (3), 66 (1) | — |
| Epicatechin gallate | 20 (0), 24 (2) | 37 (3), 43 (4) | 48 (4), 55 (5) | 77 (4), 73 (4) |
| Epigallocatechin gallate | 22 (1), 27 (2) | 46 (2), 50 (3) | 56 (1), 63 (1) | 85 (3), 82 (3) |
| 17-α-Ethinylestradiol | 31 (2), 56 (3) | 42 (2), 60 (2) | 66 (3), 80 (3) | — |
| Eugenol | 25 (2), 26 (2) | 39 (0), 41 (3) | 48 (3), 49 (1) | 73 (2), 63 (3) |
| Gallocatechin gallate | 20 (1), 29 (1) | 45 (2), 47 (2) | 62 (7), 64 (4) | 80 (6), 83 (4) |
| 4-Methylumbelliferone | 35 (3), 39 (2) | 37 (1), 40 (1) | 51 (1), 53 (2) | 69 (4), 71 (8) |
| Mycophenolic acid | 89 (3), 89 (2) | — | — | — |
| p-Nitrophenol | 37 (0), 42 (2) | 49 (2), 51 (2) | 63 (4), 66 (1) | — |
| Octyl gallate | 31 (3), 52 (7) | 37 (2), 46 (3) | 48 (6), 59 (5) | 68 (3), 75 (8) |
| Propyl gallate | 48 (2), 52 (3) | 45 (6), 48 (6) | 58 (4), 60 (4) | — |
| Quercetin | 6 (2), 13 (2) | 8 (1), 15 (1) | 18 (1), 25 (1) | 48 (1), 50 (1) |
| Silibinin | 23 (1), 22 (1) | 38 (3), 31 (6) | 56 (6), 44 (2) | 71 (3), 61 (2) |
| Tannic acid | 0 (0), 0 (0) | 0 (0), 0 (0) | 7 (1), 12 (2) | 27 (1), 30 (1) |
| Essential Oil/Extract | 50 μg/ml | 20 μg/ml | 10 μg/ml | 5 μg/ml |
| Benzoin gum powder | 21 (1), obs | 48 (1), 54 (6) | 55 (6), 54 (4) | 65 (2), 77 (1) |
| Clovebud oil | 18 (1), 12 (1) | 28 (0), 21 (1) | 38 (3), 35 (3) | 56 (5), 64 (6) |
| Silymarin | 13 (4), obs | 28 (3), 27 (2) | 53 (5), 48 (0) | 66 (7), 63 (2) |

^{a}Data are mean (SD) of triplicate determinations. The first value is for G1, the second value is for G2.
A smaller number indicates greater inhibition.
obs = obscured in HPLC trace.

TABLE 8
Comparison of inhibitor effects in different lots of liver microsomes and
pooled human jejunum microsomes.

| | % of control metabolism for indicated microsomes^{a} | | | |
|---|---|---|---|---|
| | liver (083000) | liver (032101) | liver (062101) | jejunum (HJ61) |
| Inhibitor (μM) | G1, G2 | G1, G2 | G1, G2 | G1, G2 |
| Diethylstilbestrol (10) | 58 (3), 111 (7) | 70 (2), 96 (1) | 89 (2), 83 (2) | 78 (0), 90 (4) |
| Diflunisal (50) | 43 (1), 45 (2) | 52 (1), 54 (0) | 67 (2), 57 (2) | 69 (3), 93 (6) |
| Epicatechin gallate (50) | 37 (3), 43 (4) | 47 (4), 45 (2) | 63 (4), 37 (2) | 34 (3), 40 (3) |
| Epigallocatechin gallate (50) | 46 (2), 50 (3) | 54 (2), 50 (2) | 68 (2), 41 (2) | 38 (2), 35 (4) |
| Eugenol (50) | 39 (0), 41 (3) | 65 (1), 63 (3) | 69 (4), 53 (2) | 91 (1), 96 (5) |
| Gallocatechin gallate (50) | 45 (2), 47 (2) | 29 (1), 28 (0) | 40 (3), 22 (1) | 10 (0), 10 (0) |
| Octyl gallate (50) | 37 (2), 46 (3) | 45 (1), 59 (3) | 55 (5), 45 (6) | 80 (3), 137 (9) |
| Quercetin (50) | 8 (1), 15 (1) | 25 (1), 35 (1) | 34 (3), 35 (3) | 25 (1), 37 (2) |
| Tannic Acid (10) | 27 (1), 30 (1) | 51 (4), 59 (3) | 61 (4), 54 (4) | 39 (7), 47 (8) |

^{a}Data are mean (SD) of triplicate determinations.
A smaller number indicates greater inhibition.

TABLE 9
Effects of UGT inhibitors on raloxifene glucuronidation by recombinant
UGT enzymes.

| % of control metabolism for indicated UGT enzyme^{a} | | | | |
|---|---|---|---|---|
| UGT1A1 | UGT1A3 | UGT1A8 | UGT1A9 | UGT1A10 |
| G1, G2 | G1, G2 | G1, G2 | G1, G2 | G1, G2 |

Inihibitor (μM)

| | | | | | |
|---|---|---|---|---|---|
| Diclofenac (50) | 66 (4), 73 (1) | 74 (1), 75 (5) | | 74 (2), 77 (4) | 97 (8), 93 (4) |
| Diethylstilbestrol (50) | 12 (0), 27 (0) | 14 (1), 21 (0) | | 31 (2), 245 (3) | 50 (0), 39 (1) |
| Diethylstilbestrol (25) | 26 (0), 41 (1) | 32 (3), 48 (3) | | 49 (5), 309 (13) | 74 (3) |
| Diethylstilbestrol (10) | 59 (1), 72 (1) | 65 (1), 74 (1) | 156 (7), 73 (3) | 78 (3), 290 (9) | 70 (1) |
| Diethylstilbestrol (5) | — | — | — | 86 (0), 260 (3) | — |
| Diethylstilbestrol (2) | — | — | — | 90 (0), 175 (0) | — |
| Diflunisal (50) | 31 (1), 33 (2) | 83 (2), 81 (4) | 81 (0), 126 (3) | 50 (3), 43 (3) | 90 (3), 90 (2) |
| Diflunisal (25) | 53 (0), 53 (1) | — | — | — | — |
| Epicatechin gall. (50) | 23 (3), 25 (2) | 57 (4), 42 (2) | 71 (4), 53 (4) | 48 (6), 45 (3) | 54 (3), 51 (1) |
| Epicatechin gall. (25) | 39 (2), 43 (1) | — | — | — | — |
| Epigallocatechin gall. (50) | 21 (0), 25 (1) | 26 (2), 25 (1) | 69 (3), 54 (2) | 33 (4), 22 (2) | 37 (2), 37 (1) |
| Epigallocatechin gall. (25) | 44 (1), 45 (1) | 69 (2), 64 (3) | — | 58 (1), 50 (1) | 68 (1), 73 (1) |
| 17-α-Ethinylestradiol (50) | 33 (1), 58 (3) | 45 (2), 68 (1) | | 72 (1), 81 (5) | 87 (5), 77 (1) |
| 17-α-Ethinylestradiol (25) | 53 (0), 72 (1) | — | | — | |
| Eugenol (50) | 85 (7), 79 (4) | 89 (5), 85 (2) | 107 (3), 103 (2) | 32 (1), 29 (1) | 85 (4), 81 (2) |
| Eugenol (25) | — | — | — | 56 (3), 51 (3) | — |
| Gallocatechin gall. (50) | 30 (3), 32 (4) | 39 (1), 38 (5) | 36 (3), 35 (4) | 47 (1), 44 (2) | 49 (2), 48 (1) |
| Gallocatechin gall. (25) | 52 (1), 54 (1) | 65 (1), 70 (1) | — | 72 (2), 63 (4) | 64 (0), 68 (1) |
| Mycophenolic acid (50) | 90 (5), 89 (5) | 95 (3), 87 (5) | | 81 (4), 83 (3) | 110 (2), 98 (3) |
| Octyl gallate (50) | 18 (2), 31 (3) | 12 (1), 13 (1) | 116 (9), 95 (3) | 84 (1), 70 (4) | 63 (2), 71 (2) |
| Octyl gallate (25) | 37 (1), 51 (1) | 28 (2), 31 (4) | — | — | — |
| Octyl gallate (10) | 65 (1), 75 (1) | 61 (2), 60 (2) | — | — | — |
| Propyl gallate (50) | 72 (2), 73 (4) | 55 (1), 56 (2) | | 48 (2), 51 (4) | 80 (3), 72 (2) |
| Quercetin (50) | 2 (0), 3 (1) | 28 (2), 25 (1) | 95 (2), 81 (2) | 26 (2), 24 (2) | 55 (2), 48 (1) |
| Quercetin (25) | 13 (1), 13 (1) | 53 (0), 49 (2) | | 45 (2), 39 (2) | 80 (6), 80 (5) |
| Quercetin (10) | 45 (0), 43 (1) | 81 (3), 56 (1) | | 59 (1), 56 (3) | — |
| Silibinin (50) | 18 (3), 7 (3) | 37 (3), 10 (0) | | 59 (4), 22 (0) | 56 (5), 64 (4) |
| Silibinin (25) | 46 (1), 33 (1) | 59 (2), 32 (2) | | 74 (6), 47 (4) | — |
| Silibinin (10) | 69 (3), 63 (1) | 81 (3), 56 (1) | | 93 (3), 72 (5) | — |
| Tannic acid (50) | 0.4 (0), 0.9 (0) | 3 (0), 5 (1) | — | 17 (0), 42 (5) | 10 (1), 8 (0) |
| Tannic acid (25) | 4 (1), 8 (2) | 25 (2), 38 (3) | — | 21 (2), 19 (3) | 56 (3), 53 (2) |
| Tannic acid (10) | 17 (1), 17 (1) | 48 (2), 64 (1) | 73 (1), 75 (3) | 44 (1), 45 (1) | — |
| Tannic acid (5) | 39 (2), 38 (2) | — | — | 53 (2), 76 (3) | — |
| Tannic acid (2) | 71 (3), 71 (3) | — | — | — | — |
| Inhibitor (μg/ml) | | | | | |
| Allspice berry oil (20) | 98 (1), 79 (1) | 97 (9), 86 (3) | | 21 (2), 31 (2) | 92 (6), 87 (4) |
| Allspice berry oil (10) | — | — | | 39 (1), 41 (4) | — |
| Allspice berry oil (5) | — | — | | 56 (1), 54 (3) | — |
| Benzoin powder (20) | 40 (1), 40 (0) | 27 (3), 32 (2) | | 65 (0), 87 (2) | 82 (7), 89 (3) |
| Carrot seed oil (20) | 117 (5), 120 (5) | 95 (2), 107 (5) | | 124 (2), 127 (2) | 121 (3), 110 (2) |
| Clovebud oil (20) | 90 (1), 85 (0) | 84 (1), 74 (1) | 129 (16), 115 (7) | 22 (2), 21 (1) | 68 (2), 69 (1) |
| Clovebud oil (10) | — | — | — | 32 (1), 25 (1) | — |
| Clovebud oil (5) | — | — | — | 48 (3), 39 (2) | — |
| Peppermint oil (20) | 113 (2), 113 (3) | 94 (9), 101 (8) | 123 (6), 112 (5) | 116 (0), 111 (1) | 114 (2), 102 (4) |
| Silymarin (20) | 16 (1), 11 (1) | 31 (3), 29 (2) | — | 49 (3), 46 (1) | 55 (2), 52 (4) |
| Silymarin (10) | 45 (2), 37 (1) | 48 (4), 45 (6) | | 82 (3), 61 (1) | 86 (3), 85 (3) |
| Silymarin (5) | 62 (2), 60 (2) | 68 (1), 60 (2) | | — | — |

^{a}Data are mean (SD) of triplicate determinations. A smaller number indicates greater inhibition.

TABLE 10
Inhibition of zidovudine glucuronidation in human liver microsomes (lot 062101)

| | Residual metabolism at indicated inhibitor concentration^{a} | | | | |
|---|---|---|---|---|---|
| Inhibitor | 50 μM | 100 μM | 200 μM | 500 μM | 1000 μM |
| Capsaicin | | | | 57 (4) | 34 (1) |
| Carvacrol | | 94 (1) | 75 (3) | 50 (3); 45 (0) | 20 (1) |
| Cinnamic acid | | | | 97 (3) | |
| Diclofenac | 74 (1) | 64 (2) | 52 (2) | 21 (1); 21 (1) | |
| Diethylstilbestrol | 50 (4); 44 (2) | 34 (4) | 15 (1) | 0 (0) | |
| Diflunisal | | | | 74 (2) | 43 (2) |
| Dihydrocapsaicin | | | | 50 (1) | 42 (2) |
| Estradiol | 50 (1) | 28 (1) | 24 (0) | 29 (1); 24 (2) | |
| 17-αEthinylestradiol | 54 (2) | 40 (2) | 24 (1) | 13 (1); 10 (1) | |
| Eugenol | | | | 84 (2) | 48 (4) |
| Epicatechin gallate | 80 (1) | 67 (1) | 60 (2) | 28 (1) | |
| Epigallocatechin | | | | 90 (3) | |
| Epigallocatechin gallate | 70 (1) | 60 (2) | 39 (2) | 18 (1) | |
| Gallocatechin gallate | 66 (3) | 56 (1); 59 (3) | 49 (1); 40 (2) | obs | |
| Geraniol | | 77 (5) | 62 (3) | 28 (3); 25 (0) | 9 (1) |
| Indomethacin | | | | 68 (7) | |
| Linalool | | | | 73 (5) | |
| Linoleic acid | | | | 68 (5) | |
| Menthol | | | 63 (6) | 43 (1); 37 (4) | 25 (2) |
| Menthyl acetate | | | 64 (2) | 35 (1) | 20 (1) |
| 4-Methylumbelliferone | | | | 81 (2) | |
| Naringenin | | | 64 (3) | 48 (7); 34 (4) | 15 (3) |
| N-Vanillylnonanamide | | | | 67 (1) | 40 (1) |
| Octyl gallate | | | | 54 (3) | 17 (1) |
| Propyl gallate | | | | 77 (2) | |
| Quercetin | | 94 (8) | 64 (4) | insoluble | |
| Raloxifene | | 76 (3) | 32 (4) | insoluble | |
| Retinol | | | | 76 (2) | |
| Tannic acid | 54 (2); 50 (4) | 42 (2); 37 (4) | 20 (2) | obscured | |
| Valproic acid | | | | 92 (3) | |
| Vanillin | | | | 81 (4) | |
| Vanillyl alcohol | | | | 95 (5) | |

| | Residual metabolism at indicated inhibitor concentration^{a} | | | |
|---|---|---|---|---|
| Inhibitor | 20 μg/ml | 50 μg/ml | 100 μg/ml | 200 μg/ml |
| Allspice berry oil | 73 (3) | 66 (1) | 41 (1) | 24 (2) |
| Clovebud oil | 62 (3) | 47 (2); 43 (1) | 26 (1); 24 (1) | 11 (1) |
| Peppermint oil | 61 (1) | 54 (4); 47 (2) | 34 (3); 30 (1) | 22 (1) |
| Silymarin | 86 (6) | 65 (3) | 75 (1) | 60 (1) |

^{a}Data are mean (SD, n = 3) percentage of control; a smaller number Indicates greater inhibition.

TABLE 11
Inhibition of zidovudine metabolism in pooled human
jejunal microsomes.

| Inhibitor | Residual metabolism^{a} |
|---|---|
| Diclofenac (200 μM) | 18 (2) |
| Diethylstilbestrol (50 μM) | 42 (3) |
| 17-α-Ethinylestradiol (100 μM) | 30 (1) |
| Menthol (500 μM) | 32 (3) |
| Peppermint oil (100 μg/ml) | 31 (3) |

^{a}Data are mean (SD, n = 3) percentage of control; a smaller number indicates greater inhibition

TABLE 12
Metabolism of E2 by human hepatic and jejunal microsomes.

| | Protein | Metabolism rate (pmol/min/mg)^{a} | | |
|---|---|---|---|---|
| Microsomes (lot) | μg/ml | 3-β-D | 17-β-D | E2 loss |
| Liver (032101) | 250 | 595 ± 6 | 520 ± 7 | 1,019 ± 22 |
| Liver (062101) | 250 | 370 ± 2 | 417 ± 7 | 811 ± 53 |
| Jejunum (HJ61) | 50 | 7,406 ± 183 | <LOQ | 7,999 ± 277 |

^{a}Mean ± SE of regression estimate (r² > 0.99).

TABLE 13
Relative metabolite levels after metabolism of labetalol
(100 μM) by human microsomes and UGT enzyme forms
(500 μg/ml) under identical conditions^{a.}

| Microsomes | LG1 | LG2 | LG1/LG2 |
|---|---|---|---|
| Liver | 100 (24) | 4 (1) | 25 |
| Jejunum | 98 (5) | 8 (1) | 13 |
| UGT1A9 | 38 (1) | 49 (1) | 0.8 |
| UGT2B7 | 993 (52) | 81 (3) | 12 |

^{a}Data are mean (SD, n = 3) metabolite peak area normalized to internal standard.

TABLE 14
Comparison of inhibitor effects on labetalol (50 μM)
metabolism in human liver microsomes.

| Residual metabolism at indicated | |
|---|---|
| inhibitor concentration^{a} | |
| LG1 | LG2 |

Inhibitor (μM)

| | | |
|---|---|---|
| Control | 100 (7) | 100 (6) |
| Diethylstilbestrol (10) | 66 (6) | 68 (7) |
| Diethylstilbestrol (25) | 39 (1) | 45 (3) |
| Diethylstilbestrol (50) | 23 (3) | 29 (8) |
| Diethylstilbestrol (100) | 1 (3); 5 (3) | 11 (1); 8 (1) |
| Diflunisal (50) | 92 (4) | 80 (3) |
| Diflunisal (100) | 80 (6); 86 (5) | 70 (4); 73 (2) |
| Diflunisal (500) | 43 (5) | 35 (2) |
| Epicatechin gallate (100) | 74 (1) | 86 (2) |
| Epigallocatechin (100) | 96 (7) | 87 (5) |
| Epigallocatechin gallate (100) | 55 (2) | 52 (5) |
| Eugenol (100) | 82 (2) | 75 (4) |
| Gallocatechin gallate (50) | 60 (5) | 55 (8) |
| Quercetin (50) | 78 (4) | 70 (3) |
| Quercetin (100) | 56 (8); 49 (7) | 47 (6); 43 (9) |
| Quercetin (500) | 36 (3) | 23 (1) |
| Tannic Acid (10) | 91 (2) | 88 (4) |
| Tannic Acid (25) | 56 (2) | 51 (6) |
| Tannic Acid (50) | 42 (1) | 37 (2) |
| Tannic Acid (100) | 19 (1); 21 (1) | 9 (0); 13 (3) |
| Essential oils (μg/ml) | | |
| Control | 100 (5) | 100 (8) |
| Clovebud oil (10) | 64 (3) | 62 (8) |
| Clovebud oil (50) | 19 (1) | 11 (1) |
| Clovebud oil (100) | 8 (0) | 5 (1) |
| Clovebud oil (200) | 2 (2) | 1 (0) |
| Peppermint oil (10) | 82 (5) | 85 (4) |
| Peppermint oil (50) | 46 (5) | 48 (4) |
| Peppermint oil (100) | 34 (2) | 38 (4) |
| Peppermint oil (200) | 12 (2) | 14 (2) |

^{a}Data are mean (SD, n = 3) percentage of control; a smaller number indicates greater inhibition.

TABLE 15
Comparison of inhibitor effects on E2 (50 μM) metabolism
in human liver and pooled human jejunum microsomes.

| Residual | | |
|---|---|---|
| metabolism at indicated inhibitor concentration^{a} | | |
| liver (032101) | liver (062101) | jejunum (HJ61) |
| 3-β-D; 17-β-D | 3-β-D; 17-β-D | 3-β-D; 17-β-D |

Inhibitor (μM)

| | | | |
|---|---|---|---|
| Diethylstilbestrol (1) | 85 (1), | 86 (3), 254 (2) | 86 (2), 108 (5) |
| | 361 (6) | | |
| Diethylstilbestrol (10) | 57 (4), | 71 (2), 446 (7) | 53 (4), 78 (3) |
| | 545 (32) | | |
| Diethylstilbestrol (100) | 69 (1), | — | 5 (1), 24 (4) |
| | 217 (5) | | |
| Diflunisal (100) | 49 (1), obs | 59 (2), obs | 62 (3), obs |
| Epicatechin | 27 (1), 83 (0) | 34 (1), 67 (1) | 26 (1), 61 (4) |
| gallate (50) | | | |
| Epigallocatechin | 46 (1), 82 (2) | 45 (2), 70 (1) | 32 (0), 41 (4) |
| gallate (50) | | | |
| Eugenol (50) | obs, 94 (5) | — | — |
| Eugenol (100) | obs, 89 (6) | — | — |
| Gallocatechin | 24 (1), 69 (4) | 33 (2), 55 (2) | 25 (1), 39 (1) |
| gallate (50) | | | |
| Octyl gallate (50) | 53 (1), 88 (1) | — | — |
| Octyl gallate (100) | 33 (2), 74 (2) | — | — |
| Quercetin (25) | 58 (1), 94 (3) | 56 (3), 83 (1) | 44 (5), 72 (3) |
| Quercetin (50) | 29 (1), 72 (6) | 30 (1), 69 (1) | 29 (2), 56 (2) |
| Quercetin (100) | 10 (1), 50 (3) | 19 (1), 57 (2) | 12 (1), 42 (7) |
| Tannic Acid (10) | 14 (1), 75 (3) | 26 (4), 72 (3) | 18 (1), 17 (1) |
| Essential oils(μg/ml) | | | |
| Allspice berry (50) | obs, 53 (5) | — | — |
| Allspice berry (100) | obs, 17 (2) | — | — |
| Clovebud (50) | obs, 50 (3) | — | — |
| Clovebud (100) | obs, 22 (3) | — | — |
| Peppermint (50) | 83 (2), 45 (2) | — | — |
| Peppermint (100) | 68 (2), 37 (2) | — | — |
| Silymarin (10) | 34 (3), 102 (4) | — | — |
| Silymarin (20) | 21 (2), 109 (7) | — | — |
| Silymarin (50) | 4.5 (0), 29 (2) | — | — |

^{a}Data are mean (SD, n = 3) percentage of control; a smaller number indicates greater inhibition.
obs = obscured in the HPLC trace.
— = not tested.

TABLE 16
Metabolism of 2ME2 (50 μM) by human hepatic and jejunal
microsomes.

| | Protein | Substrate loss (pmol/min/mg)^{a} |
|---|---|---|
| Microsomes (lot) | μg/ml | 2ME2 |
| Liver (032101) | 250 | 1,573 ± 243 |
| Liver (062101) | 250 | 1,035 ± 52 |
| Jejunum (HJ61) | 50 | 19,797 ± 631 |

^{a}Mean ± SE of regression estimate (r² > 0.99)

TABLE 17
Relative metabolism of 2ME2 (50 μM) by microsomes and
recombinant UGT Supersomes ® (unless indicated).

| | Relative metabolite levels^{a} | | Ratio |
|---|---|---|---|
| Microsomes | MG1 | MG2 | MG1/MG2 |
| Liver (032101) | 100 ± 5 | 28 ± 1 | 3.6 |
| Liver (062101) | 53 ± 0 | 26 ± 0 | 2.1 |
| Jejunum (HJ61) | 541 ± 18 | 1.0 ± 0.1 | 541 |
| UGT1A1 | 90 ± 5 | 1.3 ± 0.1 | 68 |
| UGT1A1 (Bacculosomes | 46 ± 1 | <LOQ | |
| UGT1A3 | 18 ± 1 | 1.7 ± 0.1 | 10.6 |
| UGT1A4 | <LOQ | 6.1 ± 0.1 | |
| UGT1A6 | <LOQ | <LOQ | |
| UGT1A7 (Bacculosomes) | 2.6 ± 0.1 | <LOQ | |
| UGT1A8 | 161 ± 2 | 1.4 ± 0.0 | 117 |
| UGT1A9 | 18 ± 0 | 2.4 ± 0.0 | 7.5 |
| UGT1A10 | 212 ± 1 | <LOQ | |
| UGT2B7 | <LOQ | 5.9 ± 0.2 | |
| UGT2B15 | <LOQ | 1.9 ± 0.1 | |

^{a}Mean ± SD (n = 3). Data are metabolite HPLC peak areas relative to the area of MG1 in human liver microsomes.

TABLE 18
Comparison of inhibitor effects on 2ME2 (50 μM)
metabolism by human liver and pooled human jejunum
microsomes.

| | | Residual metabolism | | |
|---|---|---|---|---|
| | | at indicated inhibitor concentration^{a} | | |
| | | liver (062101) | | jejunum (HJ61) |
| Inhibitor | μM | MG1 | MG2 | MG1 |
| Diclofenac | 50 | 70 (1) | 60 (1) | 100 (3) |
| | 100 | 54 (1) | 44 (3) | 97 (4) |
| Diethylstilbestrol | 1 | 88 (2) | 335 (5) | 93 (4) |
| | 10 | 72 (2) | 632 (4) | 75 (1) |
| Diflunisal | 50 | 63 (2) | 90 (5) | 91 (1) |
| | 100 | 49 (1) | 91 (1) | 82 (2) |
| Epicatechin gallate | 25 | 47 (1) | 79 (2) | 76 (4) |
| | 50 | 33 (1) | 69 (2) | 63 (1) |
| | 100 | 17 (1) | 58 (1) | 47 (2) |
| Epigallocatechin | 25 | 61 (1) | 79 (0) | 66 (2) |
| gallate | 50 | 48 (2) | 71 (1) | 50 (3) |
| | 100 | 29 (1) | 35 (1) | 35 (2) |
| Gallocatechin gallate | 10 | 56 (1) | 84 (1) | 69 (3) |
| | 25 | 33 (1) | 73 (1) | 51 (2) |
| | 50 | 21 (2) | 61 (1) | 31 (1) |
| | 100 | 9 (1) | 36 (1) | 9 (1) |
| 17-α-Ethinylestradiol | 25 | 74 (1) | 62 (1) | — |
| | 50 | 64 (2) | 41 (3) | 109 (5) |
| | 100 | 48 (0) | 4 (0) | 99 (2) |
| Eugenol | 100 | obs | 72 (1) | 104 (4) |
| Menthol | 50 | 96 (2) | 86 (1) | 98 (4) |
| | 100 | 92 (2) | 74 (3) | 125 (7) |
| Naringenin | 25 | 76 (1) | 53 (4) | — |
| | 50 | 47 (3) | 26 (5) | 78 (2) |
| | 100 | 21 (4) | 19 (0) | 79 (5) |
| Octyl gallate | 50 | 68 (3) | 94 (4) | 121 (7) |
| | 100 | 51 (0) | 65 (3) | 58 (3) |
| Quercetin | 25 | 63 (3) | 87 (3) | 56 (2) |
| | 50 | 30 (4) | 68 (3) | 44 (2) |
| | 100 | 15 (0) | 50 (2) | 19 (1) |
| Raloxifene | 5 | 77 (1) | 98 (1) | 73 (1) |
| | 10 | 63 (1) | 88 (0) | 58 (1) |
| | 25 | 42 (0) | 70 (0) | 31 (3) |
| | 50 | 32 (2) | 41 (2) | 17 (0) |
| | 100 | 24 (2) | 20 (1) | 12 (0) |
| Tannic acid | 2 | 62 (2) | 105 (3) | 63 (1) |
| | 5 | 39 (0) | 91 (5) | 49 (1) |
| | 10 | 24 (1) | 69 (6) | 42 (1) |
| | 25 | 13 (1) | 49 (7) | 5 (0) |
| | 50 | <LOQ | 28 (1) | <LOQ |
| | μg/ml | | | |
| Peppermint oil | 20 | 91 (1) | 68 (2) | — |
| | 50 | 91 (2) | 46 (2) | — |
| | 100 | 92 (7) | 37 (1) | 130 (11) |

^{a}Data are mean (SD, n = 3) percentage of control; a smaller number indicates greater inhibition.
obs = obscured in the HPLC trace.
— = not tested.

TABLE 19
Raloxifene plasma pharmacokinetics after oral administration to rats
either alone or with the indicated UGT inhibitor^{a}.

| Parameter | Control | Quercetin | Tannic Acid | Diflunisal |
|---|---|---|---|---|
| Raloxifene (mg/kg) | 10 | 10 | 10 | 10 |
| Inhibitor (mg/kg) | — | 50 | 50 | 50 |
| C_{peak,1} (ng/ml) | 21 ± 14 | 24 ± 10 | 12 ± 6 | 14 ± 8 |
| C_{peak,2} (ng/ml) | 20 ± 13^{b} | 21 ± 11 | 25 ± 8 | 19 ± 8^{b} |
| T_{peak,1} (h) | 1.5 (0.5-4.0) | 1.0 (1.0-1.5) | 1.0 (0.5-1.0) | 1.5 (1.5-3.0) |
| T_{peak,2} (h) | 4.0 (1.5-6.0)^{b} | 6.0 (3.0-8.0) | 4.5 (3.0-8.0) | 8.0 (6.0-8.0)^{b} |
| AUC₀₋₈ (ng · h/ml) | 77 ± 61 | 119 ± 51 | 127 ± 25 | 78 ± 41 |
| AUC₀₋₂₄ (ng · h/ml) | 114 ± 90 | 251 ± 63* | 208 ± 18^{c}* | 211 ± 113 |

^{a}Data are mean ± SD except Tₚₑₐₖ which are median (range). Results are for 6 rats unless indicated.
^{b}n = 4.
^{c}n = 5.
*Statistically different from the control (P < 0.05).

TABLE A1
Donor information and CYP content of different microsome lot numbers.

| | |
|---|---|
| Lot # | Donor details |

LIVER MICROSOMES

| | |
|---|---|
| 021700 | 54 y.o. Caucasian male with emphysema and a history of alcohol abuse. Daily |
| 062900 | use of high blood pressure medications (unidentified). Treated with dopamine, |
| 083000 | furosemide, cefazolin, in the hospital. C.O.D. subarachnoid hemorrhage |
| 032101 | 62 y.o. Caucasian male former smoker with a history of heart disease and |
| | hypertension. Daily use of lopid. Treated with dopamine, solumedrol and |
| | mannitol in the hospital. C.O.D. Intercerebral bleeding. |
| 062101 | 60 y.o. Caucasian male former smoker with a history of hypertension. Treated |
| | with dopamine, hydrochlorothiazide, nadolol. C.O.D. Subarachnoid |
| | hemorrhage. |

POOLED JEJUNUM MICROSOME (n = 4)

| | |
|---|---|
| HJ61 | 61 y.o. Caucasian male smoker with a history of heart murmurs and a primary |
| | astrocytoma. Lifetime exposure to pesticides. Treated with phenobarbital, |
| | sertraline, warfarin. C.O.D. Intracranial hemorrhage. |
| | 21 y.o. Caucasian male smoker and recreational drug user. Treated with |
| | ciprofloxacin, erythromycin, C.O.D. Head trauma |
| | 43 y.o. Caucasian male. No reported medications. C.O.D. Gunshot wound |
| | (head). |
| | 45 y.o. Caucasian male smoker and marijuana user. Severe psoriasis. Treated |
| | with cefazolin, dexamethasone, labetalol, lorazepam, midazolam, nimodipine, |
| | phenytoin, ranitidine, vecuronium. C.O.D. Subarachnoid hemorrhage. |

TABLE A2
Inhibition of 7-HFC (50 μM) glucuronidation in human liver microsomes (lots 021700 or 062900, 100 μg/ml, 15 min).

| | % of control metabolism (SD, n = 3) | | | | | | | | |
|---|---|---|---|---|---|---|---|---|---|
| Inhibitors | 500 μM | 200 μM | 100 μM | 50 μM | 25 μM | 10 μM | 5 μM | 2000 μM | 1000 μM |
| Acetaminophen | 103 (1) | | | | | | | | |
| Amitriptyline | 94 (1) | | | | | | | | |
| Ascorbic acid | 99 (1) | | | | | | | | |
| Ascorbyl palmitate | 11 (3), | 51 (1) | 64 (1) | | | | | | |
| | 17 (0) | | | | | | | | |
| Ascorbyl stearate | 73 (2) | | | | | | | | |
| Ascorbyl-2,6-dibutyrate | 92 (1) | | | | | | | | |
| AZT | 99 (1) | | | | | | | | |
| AZT glucuronide | 103 (1) | | | | | | | | |
| Bilirubin | 103 (1), | | | | | | | | |
| | 102 (3) | | | | | | | | |
| Capsaicin | 39 (0) | 42 (1), | 53 (1), | 69 (1) | | | | | |
| | | 44 (1) | 57 (0) | | | | | | |
| Carvacrol | 31 (1) | 63 (0) | 79 (1) | | | | | | |
| Cholesteryl palmitate | 100 (3) | | | | | | | | |
| Cinnamic acid | 94 (2) | | | | | | | | |
| Cinnamyl alcohol | 87 (1) | | | | | | | | |
| Coniferol | 87 (0) | | | | | | | | |
| Diclofenac | 30 (1), | 52 (0) | 64 (2) | | | | | | |
| | 30 (1) | | | | | | | | |
| Diethylstilbestrol | 0.1 (0) | 1 (0) | 11 (1) | 34 (2) | 57 (1) | 80 (4) | 90 (3) | | |
| Diflunisal | 37 (1), | 65 (3) | 77 (2) | | | | | | |
| | 38 (0) | | | | | | | | |
| Dihydrocapsaicin | 42 (2) | 52 (2) | 60 (3) | | | | | | |
| Epicatechin gallate | 10 (1) | 55 (3) | 67 (1) | | | | | | |
| Epigallocatechin | 81 (4) | 90 (1) | 77 (1) | | | | | | |
| Epigallocatechin gallate | 5 (1) | 37 (2) | 67 (1) | | | | | | |
| 17-β-Estradiol | 80 (2) | | | | | | | | |
| Estriol | 84 (2) | | | | | | | | |
| Estrone | 94 (3), | | | | | | | | |
| | 85 (1) | | | | | | | | |
| 17-α-Ethinylestradiol | 72 (1) | | | | | | | | |
| Ethyl palmitate | 89 (3) | | | | | | | | |
| Eugenol | 63 (1) | | | | | | | | |
| Gallic acid | 117 (2) | | | | | | | | |
| Gallocatechin gallate | 0 (0) | 25 (0) | 54 (1), 46 (2) | 68 (2) | 80 (2) | | | | |
| Geraniol | 75 (0) | | | | | | | | |
| Haloperidol | 100 (2), | | | | | | | | |
| | 116 (6) | | | | | | | | |
| Imipramine | 78 (1) | | | | | | | | |
| Indomethacin | 49 (2), | 73 (1) | 82 (0) | | | | | | |
| | 48 (1) | | | | | | | | |
| Labetalol | 95 (2) | | | | | | | | |
| Lauryl gallate | 18 (6) | 22 (1) | 43 (2), 49 (1) | 76 (2) | 92 (3) | | | | |
| Linalool | 86 (2) | | | | | | | | |
| Linoleic acid | 33 (1) | 77 (4) | 86 (3) | | | | | | |
| (L)-Menthol | 75 (2), | | | | | | | 53 (0) | 72 (1) |
| | 80 (2) | | | | | | | | |
| (−)-Menthyl acetate | 53 (2), | 72 (2) | 86 (2) | | | | | 26 (1) | 32 (1) |
| | 53 (2), | | | | | | | | |
| | 53 (1) | | | | | | | | |
| Menthol glucuronide | 94 (4) | | | | | | | | |
| 5-Methoxypsoralen (bergapten) | 97 (1), | | | | | | | | |
| | 92 (1) | | | | | | | | |
| 4-Methylumbelliferone | 45 (1), | 64 (1) | 78 (1) | | | | | | |
| | 47 (2) | | | | | | | | |
| Mycophenolic acid | 83 (2) | | | | | | | | |
| Nabumetone | 81 (3) | | | | | | | | |
| Naproxen Free Acid | 83 (1) | | | | | | | | |
| Naproxen Na salt | 82 (1) | | | | | | | | |
| α-Naphthyl palmitate | 70 (3) | | | | | | | | |
| Naringenin | 63 (1) | | | | | | | | |
| Nerol | 71 (2) | | | | | | | | |
| p-Nitrophenol | 62 (1) | | | | | | | | |
| p-Nitrophenyl palmitate | 96 (1) | | | | | | | | |
| Octyl gallate | 0.1 (0.1) | 6 (0) | 26 (1), | 65 (0) | 86 (1) | | | | |
| | | | 29 (1) | | | | | | |
| Palmitic acid | 104 (2) | | | | | | | | |
| Probenecid | 91 (1) | | | | | | | | |
| Propafenone | 108 (2) | | | | | | | | |
| Propyl gallate | 63 (0), | 71 (1) | | | | | | 32 (0) | 46 (1) |
| | 59 (1) | | | | | | | | |
| Quercetin | 52 (1), | 53 (1) | 58 (1) | | | | | | |
| | 62 (1) | | | | | | | | |
| Retinoic acid | 84 (1) | 84 (3) | 91 (2), | | | | | | |
| | | | 87 (1) | | | | | | |
| Retinol | 25 (1) | 39 (1) | 59 (1) | | | | | | |
| Retinol palmitate | 81 (4) | 82 (1) | 90 (3) | | | | | | |
| Retinyl acetate | 48 (1) | 60 (3) | 72 (1) | | | | | | |
| Salicylic acid | 64 (0), | 75 (2) | 84 (1) | | | | | 35 (1) | 47 (1) |
| | 63 (1) | | | | | | | | |
| Silibinin | 40 (2) | 54 (3) | 57 (2) | | | | | | |
| Sulindac | 85 (0) | | | | | | | | |
| Tacrolimus | 126 (4) | | | | | | | | |
| Tannic acid | 0 (0) | 0 (0) | 0 (0) | 2 (0) | 14 (1), | 47 (2), | 72 (1) | | |
| | | | | | 18 (0) | 50 (1) | | | |
| Tannic acid, USP | 0 (0) | 0.3 (0) | 0.4 (0) | 2 (1) | 16 (1) | 65 (2) | 77 (2) | | |
| Tolbutamide | 100 (0) | | | | | | | | |
| Valproic acid, free | 98 (1) | | | | | | | | |
| Valproic acid, Na salt | 102 (3) | | | | | | | | |
| Vanillin | obscured | | | | | | | | |
| Vanillyl alcohol | 42 (0), | 67 (3) | 79 (1) | | | | | | |
| | 42 (0) | | | | | | | | |
| N-Vanillylnonanamide | 48 (1) | 63 (2) | 69 (1) | | | | | | |
| Verapamil | 97 (3) | | | | | | | | |
| Vitamin K3 | 78 (2) | | | | | | | | |

| Essential oils | 500 μg/ml | 200 μg/ml | 100 μg/ml | 50 μg/ml | 20 μg/ml |
|---|---|---|---|---|---|
| Benzoin powder | 23 (7) | 40 (2) | 57 (1) | 72 (0) | |
| Peppermint oil | 31 (1) | 46 (1), 46 (1) | 57 (2), 65 (2) | 81 (0) | |
| Silymarin | 9 (6) | 26 (3) | 32 (1) | 40 (0), 39 (2) | 61 (2) |

TABLE A3
Inhibition of raloxifene (50 μM) glucuronidation in human liver
microsomes (lot 083000, 250 μg/ml, 15 min).

| | | % of control metabolism (SD, n = 3) | | | | | | |
|---|---|---|---|---|---|---|---|---|
| Inhibitor | Metab | 500 μM | 100 μM | 50 μM | 25 μM | 10 μM | 5 μM | 2 μM |
| Acetaminophen (Tylenol) | G1 | 98 (1) | | | | | | |
| | G2 | 97 (0) | | | | | | |
| Amitriptyline | G1 | 138 (6) | | | | | | |
| | G2 | 132 (5) | | | | | | |
| Ascorbyl palmitate | G1 | 26 (1) | 57 (2) | | | | | |
| | G2 | 36 (5) | 71 (4) | | | | | |
| AZT | G1 | 93 (3) | | | | | | |
| | G2 | 93 (1) | | | | | | |
| Bilirubin | G1 | 71 (4) | | | | | | |
| | G2 | 51 (6) | | | | | | |
| Capsaicin | G1 | 26 (1) | 55 (4) | | | | | |
| | G2 | 33 (3) | 69 (4) | | | | | |
| Carvacrol | G1 | 37 (1) | 64 (1) | | | | | |
| | G2 | 33 (0) | 60 (1) | | | | | |
| Diethylstilbestrol | G1 | 0 (0) | 8 (0) | 14 (1) | 36 (2) | 58 (3) | 83 (1) | 94 (6) |
| | G2 | 0 (0) | 43 (0) | 58 (2) | 100 (5) | 111 (7) | 109 (1) | 107 (5) |
| Diflunisal | G1 | 6 (1) | 30 (4) | 43 (1) | 63 (3) | | | |
| | G2 | 7 (1) | 34 (1) | 45 (2) | 66 (1) | | | |
| Diclofenac | G1 | 6 (1) | 50 (3) | 65 (1) | | | | |
| | G2 | 6 (1) | 53 (3) | 67 (1) | | | | |
| Dihydrocapsaicin | G1 | 18 (1) | 49 (1) | | | | | |
| | G2 | 26 (1) | 68 (2) | | | | | |
| Epicatechin gallate | G1 | 0 (0) | 20 (0) | 37 (3) | 48 (4) | 77 (4) | | |
| | G2 | 0 (0) | 24 (2) | 43 (4) | 55 (5) | 73 (4) | | |
| Epigallocatechin | G1 | 65 (4) | | | | | | |
| | G2 | 79 (4) | | | | | | |
| Epigallocatechin gallate | G1 | 0 (0) | 22 (1) | 46 (2) | 56 (1) | 85 (3) | | |
| | G2 | 0 (0) | 27 (2) | 50 (3) | 63 (1) | 82 (3) | | |
| 17-β-Estradiol | G1 | 39 (1) | | | | | | |
| | G2 | 56 (2) | | | | | | |
| Estriol | G1 | 46 (3) | | | | | | |
| | G2 | 60 (3) | | | | | | |
| Estrone | G1 | 75 (0) | | | | | | |
| | G2 | 101 (3) | | | | | | |
| 17-α-Ethinylestradiol | G1 | 2 (0) | 31 (2) | 42 (2) | 66 (3) | | | |
| | G2 | 6 (0) | 56 (3) | 60 (2) | 80 (3) | | | |
| Eugenol | G1 | 12 (0) | 25 (2) | 39 (0) | 48 (3) | 73 (2) | 86 (3) | |
| | G2 | 13 (2) | 26 (2) | 41 (3) | 49 (1) | 63 (3) | 74 (0) | |
| Gallocatechin gallate | G1 | 0 (0) | 20 (1) | 45 (2) | 62 (7) | 80 (6) | | |
| | G2 | 0 (0) | 29 (1) | 47 (2) | 64 (4) | 83 (4) | | |
| Haloperidol | G1 | 104 (7) | | | | | | |
| | G2 | 102 (3) | | | | | | |
| Imipramine | G1 | 127 (8) | | | | | | |
| | G2 | 111 (6) | | | | | | |
| Indomethacin | G1 | obs | | | | | | |
| | G2 | obs | | | | | | |
| Labetalol | G1 | 104 (11) | | | | | | |
| | G2 | 102 (4) | | | | | | |
| Lauryl gallate | G1 | 7 (0) | 64 (4) | | | | | |
| | G2 | 37 (1) | 85 (3) | | | | | |
| Linoleic acid | G1 | 29 (0) | 101 (8) | | | | | |
| | G2 | obs | 100 (4) | | | | | |
| (L)-Menthol | G1 | 108 (4) | | | | | | |
| | G2 | 97 (2) | | | | | | |
| Menthol glucuronide | G1 | 91 (2) | | | | | | |
| | G2 | 84 (1) | | | | | | |
| (−)-Menthyl acetate | G1 | 76 (1) | | | | | | |
| | G2 | 69 (0) | | | | | | |
| 4-Methylumbelliferone | G1 | 8 (0) | 35 (3) | 37 (1) | 51 (1) | 69 (4) | | |
| | G2 | 11 (1) | 39 (2) | 40 (1) | 53 (2) | 71 (8) | | |
| Mycophenolic acid | G1 | 43 (5) | 89 (3) | | | | | |
| | G2 | 47 (1) | 89 (2) | | | | | |
| Nabumetone | G1 | 75 (2) | | | | | | |
| | G2 | 80 (2) | | | | | | |
| Naproxen Free Acid | G1 | 81 (3) | | | | | | |
| | G2 | 78 (2) | | | | | | |
| Naringenin | G1 | obs | 34 (2) | 54 (4) | | | | |
| | G2 | obs | 40 (2) | 71 (9) | | | | |
| p-Nitrophenol | G1 | 24 (2) | 37 (0) | 49 (2) | 63 (4) | | | |
| | G2 | 29 (1) | 42 (2) | 51 (2) | 66 (1) | | | |
| Octyl gallate | G1 | | 31 (3) | 37 (2) | 48 (6) | 68 (3) | | |
| | G2 | | 52 (7) | 46 (3) | 59 (5) | 75 (8) | | |
| Probenecid | G1 | 70 (4) | | | | | | |
| | G2 | 70 (1) | | | | | | |
| Propafenone | G1 | 143 (1) | | | | | | |
| | G2 | 129 (4) | | | | | | |
| Propyl gallate | G1 | 21 (1) | 48 (2) | 45 (6) | 58 (4) | | | |
| | G2 | 33 (2) | 52 (3) | 48 (6) | 60 (4) | | | |
| Quercetin | G1 | 0 (0) | 6 (2) | 8 (1) | 18 (1) | 48 (1) | 69 (1) | 82 (6) |
| | G2 | 0 (0) | 13 (2) | 15 (1) | 25 (1) | 50 (1) | 67 (2) | 78 (3) |
| Retinoic acid | G1 | 77 (3) | | | | | | |
| | G2 | 75 (1) | | | | | | |
| Rutin | G1 | 84 (2) | 83 (10) | 88 (4) | 101 (5) | | | |
| | G2 | 61 (3) | 72 (2) | 81 (9) | 101 (4) | | | |
| Salicylic acid | G1 | 92 (2) | | | | | | |
| | G2 | 94 (2) | | | | | | |
| Silibinin | G1 | 12 (2) | 23 (1) | 38 (3) | 56 (6) | 71 (3) | 88 (0) | 87 (8) |
| | G2 | 15 (1) | 22 (1) | 31 (6) | 44 (2) | 61 (2) | 78 (1) | 83 (4) |
| Sulindac | G1 | 25 (3) | 76 (6) | | | | | |
| | G2 | 44 (2) | 79 (4) | | | | | |
| Tannic acid, USP | G1 | | 0 (0) | 0 (0) | 7 (1) | 27 (1) | 46 (3) | 80 (7) |
| | G2 | | 0 (0) | 0 (0) | 12 (2) | 30 (1) | 48 (1) | 74 (5) |
| Tolbutamide | G1 | 98 (7) | | | | | | |
| | G2 | 91 (2) | | | | | | |
| Valproic acid, free | G1 | 101 (7) | | | | | | |
| | G2 | 98 (1) | | | | | | |
| N-Vanillylnonanamide | G1 | 14 (1) | 48 (1) | | | | | |
| | G2 | 17 (2) | 62 (1) | | | | | |

| Essential oils | | 500 μg/ml | 100 μg/ml | 50 μg/ml | 20 μg/ml | 10 μg/ml | 5 μg/ml |
|---|---|---|---|---|---|---|---|
| Benzoin powder | G1 | 0 (0) | 5 (1) | 21 (1) | 48 (1) | 55 (6) | 65 (2) |
| | G2 | obs | obs | obs | 54 (6) | 54 (4) | 77 (1) |
| Clovebud oil | G1 | | 12 (0) | 18 (1) | 28 (0) | 38 (3) | 56 (5) |
| | G2 | | 8 (1) | 12 (1) | 21 (1) | 35 (3) | 64 (6) |
| Peppermint oil | G1 | 52 (1) | 113 (3) | | | | |
| | G2 | 41 (1) | 92 (3) | | | | |
| Silymarin | G1 | obs | 0 (0) | 13 (4) | 28 (3) | 53 (5) | 66 (7) |
| | G2 | obs | 0 (0) | obs | 27 (2) | 48 (0) | 63 (2) |

TABLE A4
Inhibition of raloxifene glucuronidation in insect microsomes expressing
recombinant human UGT enzymes.

| | | UGT1A1* | | | | | UGT1A3* | | |
|---|---|---|---|---|---|---|---|---|---|
| Inhibitor | Metab | 50 μM | 25 μM | μM | 5 μM | 2 μM | 50 μM | 25 μM | 10 μM |
| Diclofenac | G1 | 66 (4) | | | | | 74 (1) | | |
| | G2 | 73 (1) | | | | | 75 (5) | | |
| Diethylstilbestrol | G1 | 12 (0) | 26 (0) | 59 (1) | | | 14 (1) | 32 (3) | 65 (1) |
| | G2 | 27 (0) | 41 (1) | 72 (1) | | | 21 (0) | 48 (3) | 74 (1) |
| Diflunisal | G1 | 31 (1) | 53 (0) | | | | 83 (2) | | |
| | G2 | 33 (2) | 53 (1) | | | | 81 (4) | | |
| Epicatechin gallate | G1 | 23 (3) | 39 (2) | 67 (2) | | | 57 (4) | | |
| | G2 | 25 (2) | 43 (1) | 68 (2) | | | 42 (2) | | |
| Epigallocatechin | G1 | 21 (0) | 44 (1) | 70 (1) | | | 26 (2) | 69 (2) | |
| gallate | | | | | | | | | |
| | G2 | 25 (1) | 45 (1) | 74 (2) | | | 25 (1) | 64 (3) | |
| 17-α- | G1 | 33 (1) | 53 (0) | 81 (0) | | | 45 (2) | | |
| Ethinylestradiol | | | | | | | | | |
| | G2 | 58 (3) | 72 (1) | 91 (1) | | | 68 (1) | | |
| Eugenol | G1 | 85 (7) | | | | | 89 (5) | | |
| | G2 | 79 (4) | | | | | 85 (2) | | |
| Gallocatechin | G1 | 30 (3) | 52 (1) | 79 (1) | | | 39 (1) | 64 (1) | |
| gallate | | | | | | | | | |
| | G2 | 32 (4) | 54 (1) | 76 (3) | | | 38 (5) | 70 (1) | |
| Mycophenolic | G1 | 90 (5) | | | | | 95 (3) | | |
| acid | | | | | | | | | |
| | G2 | 89 (5) | | | | | 87 (5) | | |
| Octyl gallate | G1 | 18 (2) | 37 (1) | 65 (1) | | | 12 (1) | 28 (2) | 61 (2) |
| | G2 | 31 (3) | 51 (1) | 75 (1) | | | 13 (1) | 31 (4) | 60 (2) |
| Propyl gallate | G1 | 72 (2) | | | | | 55 (1) | 72 (1) | |
| | G2 | 73 (4) | | | | | 56 (2) | 79 (4) | |
| Quercetin | G1 | 2 (0) | 13 (1) | 45 (0) | 71 (2) | 89 (2) | 28 (2) | 52 (0) | 72 (5) |
| | G2 | 3 (1) | 13 (1) | 43 (1) | 70 (3) | 90 (2) | 25 (1) | 49 (2) | 73 (2) |
| Silibinin | G1 | 18 (3) | 46 (1) | 69 (3) | | | 37 (3) | 58 (2) | 81 (3) |
| | G2 | 7 (3) | 33 (1) | 63 (1) | | | 10 (0) | 32 (2) | 56 (1) |
| Tannic acid | G1 | 0.4 (0) | 4 (1) | 17 (1) | 39 (2) | 71 (3) | 3 (0) | 25 (2) | 48 (2) |
| | G2 | 0.9 | 8 (2) | 17 (1) | 38 (2) | 71 (3) | 5 (1) | 38 (3) | 64 (1) |
| | | (0.1) | | | | | | | |

| | | 20 | 10 | 5 | 2 | 1 | 20 | 10 | 5 |
|---|---|---|---|---|---|---|---|---|---|
| Essential oils | | μg/ml | μg/ml | μg/ml | μg/ml | μg/ml | μg/ml | μg/ml | μg/ml |
| Allspice berry oil | G1 | 98 (1) | | | | | 97 (9), 95 (9) | | |
| | G2 | 79 (1) | | | | | 86 (3) | | |
| Benzoin powder | G1 | 40 (1) | | | | | 27 (3) | | |
| | G2 | 40 (0) | | | | | 32 (2) | | |
| Carrot seed oil | G1 | 117 (5) | | | | | 95 (2), 95 (6) | | |
| | G2 | 120 (5) | | | | | 107 (5) | | |
| Clovebud oil | G1 | 90 (1) | | | | | 84 (1) | | |
| | G2 | 85 (0) | | | | | 74 (1) | | |
| Peppermint oil | G1 | 113 (2) | | | | | 94 (9), | | |
| | | | | | | | 91 (12) | | |
| | G2 | 113 (3) | | | | | 101 (8) | | |
| Silymarin | G1 | 16 (1) | 45 | 62 (2) | | | 31 (3) | 48 (4) | 68 (1) |
| | | | (2) | | | | | | |
| | G2 | 11 (1) | 37 | 60 (2) | | | 29 (2) | 45 (6) | 60 (2) |
| | | | (1) | | | | | | |

| | UGT1A9* | | | | | UGT1A10† |
|---|---|---|---|---|---|---|
| Inhibitor | 50 μM | 25 μM | 10 μM | 5 μM | 2 μM | 50 μm |
| Diclofenac | 74 (2) | | | | | 97 (8) |
| | 77 (4) | | | | | 93 (4) |
| Diethylstilbestrol | 31 (2) | 49 (5) | 78 (3) | 86 (0) | 90 (0) | 50 (0) |
| | 245 (3) | 309 | 290 (9) | 260 (3) | 175 (0) | 39 (1) |
| | | (13) | | | | |
| Diflunisal | 50 (3) | | | | | 90 (3) |
| | 43 (3) | | | | | 90 (2) |
| Epicatechin gallate | 48 (6) | | | | | 54 (3) |
| | 45 (3) | | | | | 51 (1) |
| Epigallocatechin | 33 (4) | 58 (1) | | | | 37 (2) |
| gallate | | | | | | |
| | 22 (2) | 50 (1) | | | | 37 (1) |
| 17-α- | 72 (1) | | | | | 87 (5) |
| Ethinylestradiol | | | | | | |
| | 81 (5) | | | | | 77 (1) |
| Eugenol | 32 (1) | 56 (3) | | | | 85 (4) |
| | 29 (1) | 51 (3) | | | | 81 (2) |
| Gallocatechin | 47 (1) | 72 (2) | | | | 49 (2) |
| gallate | | | | | | |
| | 44 (2) | 63 (4) | | | | 48 (1) |
| Mycophenolic | 81 (4) | | | | | 110 (2) |
| acid | | | | | | |
| | 83 (3) | | | | | 99 (3) |
| Octyl gallate | 84 (1) | | | | | 63 (2) |
| | 70 (4) | | | | | 71 (2) |
| Propyl gallate | 48 (2) | | | | | 80 (3) |
| | 51 (4) | | | | | 72 (2) |
| Quercetin | 26 (2) | 45 (2) | 59 (1) | 83 (1) | | 55 (2) |
| | 24 (2) | 39 (2) | 56 (3) | 85 (5) | | 48 (1) |
| Silibinin | 59 (4) | 74 (6) | 93 (3) | | | 56 (5) |
| | 22 (0) | 47 (4) | 72 (5) | | | 64 (4) |
| Tannic acid | 17 (0) | 21 (2) | 44 (1) | 53 (2) | | 10 (1) |
| | 42 (5) | 19 (3) | 45 (1) | 76 (3) | | 8 (0) |

| | 20 | 10 | 5 | 2 | 1 | 20 |
|---|---|---|---|---|---|---|
| Essential oils | μg/ml | μg/ml | μg/ml | μg/ml | μg/ml | μg/ml |
| Allspice berry oil | 21 (2) | 39 (1) | 56 (1) | | | 92 (6) |
| | 31 (2) | 41 (4) | 54 (3) | | | 87 (4) |
| Benzoin powder | 65 (0) | | | | | 82 (7) |
| | 87 (2) | | | | | 89 (3) |
| Carrot seed oil | 124 (2) | | | | | 121 (3) |
| | 127 (2) | | | | | 110 (2) |
| Clovebud oil | 22 (2) | 32 (1) | 48 (3) | 74 (4) | | 68 (2) |
| | 21 (1) | 25 (1) | 39 (2) | 65 (2) | | 69 (1) |
| Peppermint oil | 116 (0) | | | | | 114 (2) |
| | 111 (1) | | | | | 102 (4) |
| Silymarin | 49 (3) | 82 (3) | | | | 55 (2) |
| | 46 (1) | 61 (1) | | | | 52 (4) |

*Raloxifene 50 μM, 250 μg/ml microsomal protein (Supersomes), 1 mM UDPGA, 15 min
†Raloxifene 50 μM, 500 μg/ml microsomal protein (Bacculosomes), 1 mM UDPGA, 15 min

What is claimed is:

1. A method of increasing the bioavailability of an orally administered pharmaceutical compound, which method comprises:
orally coadministering to a mammal in need of treatment by said pharmaceutical compound, (1) said pharmaceutical compound and (2) an inhibitor of a UDP-glucuronosyltransferase enzyme normally present in said mammal, wherein said pharmaceutical compound is selected from the group consisting of raloxifene, 2-methoxyestradiol, irinotecan, SN-38, estradiol, labetalol, dilevalol, zidovudine (AZT) or morphine, wherein said inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, or silymarin, said inhibitor being present in an amount sufficient to provide bioavailability of said pharmaceutical compound in the presence of the inhibitor that is greater than the bioavailability of said pharmaceutical compound in the absence of said inhibitor.

2. The method of claim 1, wherein the inhibitor is coadministered in an amount sufficient to reduce the glucuronidation of the pharmaceutical compound by 50% in vitro.

3. The method of claim 1, wherein the amount of the inhibitor used is sufficient to produce a concentration of the inhibitor in the lumen of the gut of the mammal of at least 0.1 times a Kᵢ or apparent Kᵢ of the inhibitor for glucuronidation of the pharmaceutical compound.

4. The method of claim 1, wherein bioavailability of the pharmaceutical compound in the presence of the inhibitor is greater than bioavailability of the compound in the absence of the inhibitor by at least 10% of the difference between bioavailability in the absence of the inhibitor and complete oral bioavailability.

5. The method of claim 1, wherein bioavailability of the pharmaceutical compound in the presence of the inhibitor is greater than bioavailability of the compound in the absence of the inhibitor by at least 50% of the difference between bioavailability in the absence of the inhibitor and complete oral bioavailability.

6. The method of claim 1, wherein bioavailability of the pharmaceutical compound in the presence of the inhibitor is greater than bioavailability of the compound in the absence of the inhibitor by at least 75% of the difference between bioavailability in the absence of the inhibitor and complete oral bioavailability.

7. The method of claim 1, wherein said pharmaceutical compound is raloxifene and said inhibitor is selected from the group consisting of tannic acid, quercetin, eugenol, silibinin, octyl gallate, epicatechin gallate, epigallocatechin gallate, gallocatechin gallate and benzoin powder.

8. The method of claim 1, wherein said pharmaceutical compound is zidovudine (AZT) and said inhibitor is selected from the group consisting of tannic acid, gallocatechin gallate, clovebud oil, menthol, menthyl acetate, geraniol, capsaicin, dihydrocapsaicin, N-vanillylnonanamide and peppermint oil.

9. The method of claim 1, wherein said pharmaceutical compound is estradiol and said inhibitor is selected from the group consisting of tannic acid, quercetin, epicatechin gallate, epigallocatechin gallate, gallocatechin gallate, silimarin, allspice berry oil,; clovebud oil, and peppermint oil.

10. The method of claim 1, wherein said pharmaceutical compound is 2-methoxyestradiol and said inhibitor is selected from the group consisting of tannic acid, quercetin, epicatechin gallate, epigallocatechin gallate, gallocatechin gallate, naringenin and peppermint oil.

11. The method of claim 1, wherein said inhibitor is tannic acid and said pharmaceutical compound is selected from the group consisting of raloxifene, zidovudine, estradiol, and 2-methoxyestradiol.

12. The method of claim 1, wherein said inhibitor is quercetin and said pharmaceutical compound is selected from the group consisting of raloxifene and 2-methoxyestradiol.

13. A method of formulating an oral pharmaceutical composition, which method comprises: admixing a pharmaceutical compound, a pharmaceutical carrier suitable for oral administration, and a UDP-glucuronosyltransferase inhibitor, wherein said pharmaceutical compound is selected from the group consisting of raloxifene, 2-methoxyestradiol, irinotecan, SN-38, estradiol, labetalol, dilevalol, zidovudine (AZT) and morphine, wherein said inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin, said inhibitor being present in an amount sufficient to provide bioavailability of said pharmaceutical compound in the presence of the inhibitor that is greater than the bioavailability of said pharmaceutical compound in the absence of said inhibitor.

14. The method of claim 13, wherein the amount of inhibitor administered is sufficient to reduce the glucuronidation of the compound by 50% in vitro.

15. The method of claim 13, wherein the amount of the inhibitor used is sufficient to produce a concentration of the inhibitor in the lumen of the gut of the mammal of at least 0.1 times a Kᵢ or apparent Kᵢ of the inhibitor for glucuronidation of the pharmaceutical compound.

16. The method of claim 13, wherein bioavailability of the pharmaceutical compound in the presence of the inhibitor is greater than bioavailability of the compound in the absence of the inhibitor by at least 10% of the difference between bioavailability in the absence of the inhibitor and complete oral bioavailability.

17. The method of claim 13, wherein bioavailability of the pharmaceutical compound in the presence of the inhibitor is greater than bioavailability of the compound in the absence of the inhibitor by at least 50% of the difference between bioavailability in the absence of the inhibitor and complete oral bioavailability.

18. The method of claim 13, wherein bioavailability of the pharmaceutical compound in the presence of the inhibitor is greater than bioavailability of the compound in the absence of the inhibitor by at least 75% of the difference between bioavailability in the absence of the inhibitor and complete oral bioavailability.

19. The method of claim 13, wherein said pharmaceutical compound is raloxifene and said inhibitor is selected from the group consisting of tannic acid, quercetin, eugenol, silibinin, octyl gallate, epicatechin gallate, epigallocatechin gallate, gallocatechin gallate and benzoin powder.

20. The method of claim 13, wherein said pharmaceutical compound is zidovudine (AZT) and said inhibitor is selected from the group consisting of tannic acid, gallocatechin gallate, clovebud oil, menthol, menthyl acetate, geraniol, capsaicin, dihydrocapsaicin, N-vanillylnonanamide and peppermint oil.

21. The method of claim 13, wherein said pharmaceutical compound is estradiol and said inhibitor is selected from the group consisting of tannic acid, quercetin, epicatechin gallate, epigallocatechin gallate, gallocatechin gallate, silimarin, allspice berry oil, clovebud oil, and peppermint oil.

22. The method of claim 13, wherein said pharmaceutical compound is 2-methoxyestradiol and said inhibitor is selected from the group consisting of tannic acid, quercetin, epicatechin gallate, epigallocatechin gallate, gallocatechin gallate, naringenin and peppermint oil.

23. The method of claim 13, wherein said inhibitor is tannic acid and said pharmaceutical compound is selected from the group consisting of raloxifene, zidovudine, estradiol, and 2-methoxyestradiol.

24. The method of claim 13, wherein said inhibitor is quercetin and said pharmaceutical compound is selected from the group consisting of raloxifene and 2-methoxyestradiol.

25. The pharmaceutical composition produced by the process of claim 13.

26. A method of increasing bioavailability of the active compound of an existing oral pharmaceutical composition comprising a pharmaceutical compound, which method comprises: reformulating the existing composition to provide a reformulated oral composition by admixing the pharmaceutical compound with a UDP-glucuronosyltransferase inhibitor, wherein said pharmaceutical compound is selected from the group consisting of raloxifene, 2-methoxyestradiol, irinotecan, SN-38, estradiol, labetalol, dilevalol, zidovudine (AZT) and morphine, wherein said inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin, said inhibitor being present in an amount sufficient to provide bioavailability of said pharmaceutical compound when administered in the reformulated composition greater than the bioavailability of said pharmaceutical compound when administered in the existing pharmaceutical composition.

27. The method of claim 26, wherein the amount of inhibitor administered is sufficient to reduce the glucuronidation of the compound by 50% in vitro.

28. The method of claim 26, wherein the amount of the inhibitor used is sufficient to produce a concentration of the inhibitor in the lumen of the gut of the mammal of at least 0.1 times a Kᵢ or apparent Kᵢ of the inhibitor for glucuronidation of the pharmaceutical compound.

29. The method of claim 26, wherein bioavailability of said pharmaceutical compound when administered in the reformulated composition is greater than bioavailability of said pharmaceutical compound when administered in the existing pharmaceutical composition by at least 10% of the difference between bioavailability in the absence of the inhibitor and complete oral bioavailability.

30. The method of claim 26, wherein bioavailability of said pharmaceutical compound when administered in the reformulated composition is greater than bioavailability of said pharmaceutical compound when administered in the existing pharmaceutical composition by at least 50% of the difference between bioavailability in the absence of the inhibitor and complete oral bioavailability.

31. The method of claim 26, wherein bioavailability of said pharmaceutical compound when administered in the reformulated composition is greater than bioavailability of said pharmaceutical compound when administered in the existing pharmaceutical composition by at least 75% of the difference between bioavailability in the absence of the inhibitor and complete oral bioavailability.

32. The method of claim 26, wherein said pharmaceutical compound is raloxifene and said inhibitor is selected from the group consisting of tannic acid, quercetin, eugenol, silibinin, octyl gallate, epicatechin gallate, epigallocatechin gallate, gallocatechin gallate and benzoin powder.

33. The method of claim 26, wherein said pharmaceutical compound is zidovudine (AZT) and said inhibitor is selected from the group consisting of tannic acid, gallocatechin gallate, clovebud oil, menthol, menthyl acetate, geraniol, capsaicin, dihydrocapsaicin, N-vanillylnonanamide and peppermint oil.

34. The method of claim 26, wherein said pharmaceutical compound is estradiol and said inhibitor is selected from the group consisting of tannic acid, quercetin, epicatechin gallate, epigallocatechin gallate, gallocatechin gallate, silimarin, allspice berry oil, clovebud oil, and peppermint oil.

35. The-method of claim 26, wherein said pharmaceutical compound is 2-methoxyestradiol and said inhibitor is selected from the group consisting of tannic acid, quercetin, epicatechin gallate, epigallocatechin gallate, gallocatechin gallate, naringenin and peppermint oil.

36. The method of claim 26, wherein said inhibitor is tannic acid and said pharmaceutical compound is selected from the group consisting of raloxifene, zidovudine, estradiol, and 2-methoxyestradiol.

37. The method of claim 26, wherein said inhibitor is quercetin and said pharmaceutical compound is selected from the group consisting of raloxifene and 2-methoxyestradiol.

38. The pharmaceutical composition produced by the process of claim 26.
